SEQUENCE LISTING (8 sequences)

SEQ ID NO: 1 - RNA, 20 nt, synthetic construct
  source 1..20: mol_type = other RNA
ttcagaatcaaaggcatggg

SEQ ID NO: 2 - DNA, 127 bp, synthetic construct
  source 1..127: mol_type = other DNA
tcctcagaattgctgaggatcaactcttggcgagggtggaagacggcacaagatacattgttgtaatggccccggcaggtatcaactttccaggcttttgattctgaaggacaaaaagaattaggtc

SEQ ID NO: 3 - DNA, 22 bp, synthetic construct
  source 1..22: mol_type = other DNA
accactccctgctgctttgcct

SEQ ID NO: 4 - DNA, 22 bp, synthetic construct
  source 1..22: mol_type = other DNA
ggcacacacttggcggttcctt

SEQ ID NO: 5 - DNA, 24 bp, synthetic construct
  source 1..24: mol_type = other DNA
agtgctgccgtcattttctgcctc

SEQ ID NO: 6 - DNA, 25 bp, synthetic construct
  source 1..25: mol_type = other DNA
gcaggataggctcgcagggatgatt

SEQ ID NO: 7 - DNA, 21 bp, synthetic construct
  source 1..21: mol_type = other DNA
aggtcggtgtgaacggatttg

SEQ ID NO: 8 - DNA, 23 bp, synthetic construct
  source 1..23: mol_type = other DNA
tgtagaccatgtagttgaggtca

(19) United States
(12) Patent Application Publication
(10) Pub. No.: US 2026/0216376 A1
(43) Pub. Date: Jul. 30, 2026

(54) HAQ STING AS A TREATMENT FOR COPA SYNDROME

(71) Applicant: The Regents of the University of California, Oakland, CA (US)

(72) Inventor: Anthony SHUM, San Francisco, CA (US)

(21) Appl. No.: 19/462,786

(22) Filed: Jan. 28, 2026

Related U.S. Application Data
(60) Provisional application No. 63/750,567, filed on Jan. 28, 2025.

(51) Int. Cl. A61K 48/00; A61P 37/02; C12N 15/86; C12Q 1/6883

(52) U.S. Cl. CPC A61K 48/005; A61P 37/02; C12N 15/86; C12Q 1/6883; C12N 2740/15043; C12Q 2600/106

(57) ABSTRACT

The methods provided herein are, inter alia, useful for the treatment of a STING-mediated disease in a coatomer protein subunit alpha (COPA) syndrome patient who carries a genetic variant STING allele. By introducing a gene therapy targeting the genetic variant STING allele in said COPA syndrome patient, the patient is treated for the STING-mediated disease.

### RELATED APPLICATION DATA

This application claims the benefit of priority under 35 U.S.C. § 119(e) of the U.S. Patent Application No. 63/750,567, filed on Jan. 28, 2025, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. RO1 AI137249 and 1R01 AI168299 awarded by the National Institute of Health. The government has certain rights in the invention.

### SEQUENCE LISTING

The material in the accompanying Sequence Listing is hereby incorporated by reference in its entirety and for all purposes. The accompanying file, named “048536-802001US_SL_ST26.xml” was created on Jan. 28, 2026, and is 8,085 bytes in size.

### BACKGROUND

COPA Syndrome is a rare autosomal-dominant inborn error of immunity defined by childhood onset of interstitial lung disease (ILD), high-titer autoantibodies, and inflammatory arthritis (Watkin et al., 2015). COPA Syndrome has reduced penetrance, with 15-30% of individuals with pathogenic coatomer protein alpha (COPA) mutations completely lacking clinical signs and symptoms of disease (Watkin et al., 2015; Fremond and Nathan, 2021; Simchoni et al., 2023). There is a need in the art for effective treatment of COPA syndrome and the methods provided herein, inter alia, address this and other needs in the art.

### BRIEF SUMMARY

In an aspect is provided, a method of treating a Stimulator of Interferon Genes (STING)-mediated disease in a subject having or being at risk of having coatomer protein subunit alpha (COPA) syndrome, the method including administering a therapeutically effective amount of a STING gene therapy to the subject thereby treating the STING-mediated disease in the subject.

### BRIEF DESCRIPTION OF THE DRAWINGS

FIG. 1 shows non-penetrance of COPA syndrome co-segregates with HAQ STING. The pedigrees of families A through F are shown with variant information provided for COPA, either wildtype (WT) in black or familial mutant in red, and, where known, STING, with major allele (232R) or 232H allele in black and HAQ in blue. Families with HAQ STING demonstrate incomplete penetrance, in contrast to family F which lacks HAQ STING and demonstrates complete penetrance. Asterisks indicate study participants. See also Extended Data FIG. 1 representing an additional 2 families with HAQ STING and incomplete penetrance, an additional completely penetrant family without HAQ STING, and 3 sporadic patients who all lack HAQ STING.

FIGS. 2A-2D show unaffected COPA mutation carriers lack pulmonary disease and elevated type I interferon levels. FIG. 2A: Chest computed tomography (CT) scan of affected patient C. II.1 at age 19 (on left) shows ground glass opacities (GGOs), reticulation, and cystic changes, none of which are seen in her father C. I.1, an unaffected carrier of a COPA mutation carrier, at age 51. FIG. 2B: CT scan of affected patient B. III.1 at age 15 months (top left) shows centrilobular nodules and GGOs, CT scans of her affected father B. II.1 show progression of reticulation and traction bronchiectasis from age 40 (bottom left) to age 51 (bottom right), along with development of cystic changes. This contrasts with an essentially normal CT of her grandmother B. I.2, an unaffected carrier of a COPA mutation, from age 78 (top right). FIG. 2C: Interferon scores calculated from the median fold change in relative quantification values for a set of six interferon stimulated genes (IFI27, IFI44L, IFIT1, ISG1S, RSAD2, SIGLEC1)(Rice et al., 2013). Bars reflect medians, with scores from affected patients (n=12 unique, 20.85; IQR 11.1-64.86) significantly higher than in healthy controls (n=12 unique, 1.00; IQR 0.6275-1.268; p<0.0001) or unaffected carriers of COPA mutations (n=8 unique, 2.075; IQR 1.184-14.80; p=0.0442). Unaffected carriers of COPA mutations did not differ from controls (p=0.2355) (Kruskal-Wallis test with Dunn's multiple comparisons). Results for five affected patients were previously published (Volpi et al., 2018). FIG. 2D: Serum interferon activity (Units/mL) measured using HEK-Blue reporter cell line. Bars reflect medians, with values from patients (n=8 unique, 4.525; IQR 2.113-5.969) significantly higher than in healthy controls (n=12, 1.853, IQR 0.9295-2.980; p=0.0169). Unaffected carriers of COPA mutations did not differ from controls (n=5 unique, 2.190; IQR 1.062-3.270, p>0.9999) (Kruskal-Wallis test with Dunn's multiple comparisons). For all panels: * indicates p<0.05, **p<0.01, ****p<0.0001, and ns not significant (p>0.05).

FIGS. 3A-3C show the HAQ STING allele co-segregated perfectly with clinical non-penetrance. FIG. 3A: The observed frequencies of HAQ STING among individuals with COPA mutations resembled the frequencies expected based on the 1000 Genome Project (Yi et al., 2013), however, no affected patients had this allele while all unaffected carriers had one copy. FIG. 3B: The observed frequencies of 232H STING among individuals with COPA mutations was above the frequencies expected based on the 1000 Genome Project, with higher frequencies seen in patients but not unaffected carriers. FIG. 3C: Variants identified using exome sequencing of four unrelated kindreds of individuals with COPA mutations. Counts shown for all variants identified per family, then sequentially filtered to focus on protein-coding variants with an allele frequency of under 50% that were unique to unaffected carriers of COPA mutations. Only the variants of HAQ STING, i.e. the R71H, G230A, and R293Q single nucleotide polymorphisms, were common across all unaffected COPA mutation carriers.

FIGS. 4A-4D show HAQ STING abrogates steady state STING activation induced by COPA mutation or depletion. FIG. 4A: Interferon scores calculated from the median fold change in relative quantification values for six ISGs (IFI27, IFI44L, IFIT1, ISG1S, RSAD2, SIGLEC1)(Rice et al., 2013) in HEK293T cells with wildtype or CRISPR edited mutant (E241K) COPA transduced with 232R or HAQ STING. Samples from 232R transduced E241K/E241K COPA cells had significantly elevated interferon scores relative to unmanipulated HEK293T cells (Friedman test p=0.181, Dunn's multiple comparisons p=0.01). Bars represent mean and standard error of the mean. FIG. 4B: Confocal microscopy of Sting1 knockout mouse embryonic fibroblasts (MEFs) reconstituted with human EGFP-tagged 232R, 232H, or HAQ STING and treated with Copa-specific siRNA. Both 232R and 232H STING colocalized with HaloTag7-Rab6 (trans-Golgi marker) in Copa knockdown MEFs, while HAQ STING did not. Pearson's correlation of overlap between STING and Rab6 in the presence of control or Copa siRNA presented to right. FIG. 4C: Sting1 knockout MEFs were reconstituted with human FLAG-232R plus EGFP-tagged 232R, 232H, or HAQ STING. Copa depletion triggered transcription of the inflammatory chemokine Cxcl10 in MEFs reconstituted with 232R/232R and 232R/232H, while those reconstituted with 232R/HAQ were protected. Bars represent mean and error bars standard deviation for results from three independent experiments. Statistical testing was performed via Student's unpaired t-test. FIG. 4D: Immunoblotting for TBK-1 phosphorylation status was performed for 232R or HAQ transduced primary human lung fibroblasts, demonstrating increased phosphorylation (activation) in 232R transduced patient-derived cells and normalization of phosphorylation in HAQ transduced fibroblasts. Bars represent means and error bars represent standard error of the mean for results from three independent experiments. Statistical testing was performed via 2-way ANOVA (p=0.0166 for impact of STING in patient cells only). Sidik's multiple comparison testing plotted. For all panels: * indicates p<0.05, **p<0.01, ***p<0.001, and ns not significant (p>0.05).

FIGS. 5A-5E show clinical data and STING expression in subject peripheral blood mononuclear cells. FIG. 5A: The pedigrees of families G through I and 3 sporadic patients are shown with variant information provided for COPA, either wild-type (WT) in black or familial mutant in red, and, where known, STING, with major allele (232R) or 232H allele in black and HAQ in blue. Individuals with COPA mutations plus HAQ STING demonstrate clinical non-penetrance. FIG. 5B: Kaplan-Meier analysis of age at symptom onset shows affected patients develop symptoms significantly before the age of most recent evaluations of unaffected carriers (log-rank test, p<0.0001). FIG. 5C: Kaplan-Meier analysis of overall survival shows higher survival in unaffected carriers relative to affected patients (log-rank test, p=0.019). While the proportional hazards assumption for symptom onset cannot be empirically validated in unaffected carriers due to complete censorship, prior to our identification of HAQ STING as the mediator of disease non-penetrance all individuals with COPA mutations would be expected to have equal likelihood of developing disease. STING1 expression relative to GAPDH was evaluated in PBMCs of 5 patients, 4 unaffected carriers, and 8 healthy controls, including 4 healthy controls with a single copy of HAQ STING. Bars reflect medians. No differences were seen either (FIG. 5D) based on disease status or (FIG. 5E) based on presence of a single copy of the HAQ allele. Statistical testing was performed via Kruskal-Wallis test with Dunn's multiple comparisons for FIG. 5D, Mann-Whitney test for FIG. 5E. ns indicates not significant (p>0.05).

FIGS. 6A-6G show data for cell line models. FIG. 6A: STING1 expression was evaluated in HEK293T cells with wildtype or CRISPR edited mutant (E241K) COPA transduced with 232R or HAQ STING. Relative expression to parental 293T cell lines was determined and divided by 29.9 to obtain fold stimulation of STING1 relative to the ancestral 293 line per Reus et. al. (Reus et al., 2020). Bars represent medians of four independent experiments. Statistical testing was performed via Friedman test (p=0.0069) with Dunn's multiple comparisons within COPA genotypes (p>0.999). ns indicates not significant (p>0.05). FIG. 6B: Sting1 knockout mouse embryonic fibroblasts (MEFs) were reconstituted with human 232R, 232H, or HAQ STING and transduced with wildtype (WT) or mutant (E241K) Copa. Transcription of inflammatory chemokines Cxcl10 (top) and CcI5 (bottom) was increased in the presence of 232R and 232H but not HAQ STING. FIG. 6C: Sting1 knockout MEFs reconstituted with human 232R, 232H, or HAQ STING and treated with Copa-specific siRNA showed increased cytokine transcription in the presence of 232R and 232H but not HAQ STING. FIG. 6D: Sting1 and cyclic GMP AMP synthetase (cGAS) double knock out MEFs were reconstituted with human 232R, 232H, or HAQ STING and treated with Copa-specific siRNA. Cytokine activation seen in Copa knockdown cells transduced with 232R or 232H STING is not dependent on cGAS production of cyclic GMP AMP (cGAMP). Bars represent mean and error bars standard deviation of 3 independent experiments. Statistical testing performed via Student's unpaired t-test. ** indicates p<0.01, *** indicates p<0.001. FIG. 6E: Sting1 knockout mouse embryonic fibroblasts (MEFs) were reconstituted with human 232R plus 232R (major allele), 232H, or HAQ STING. Endogenous expression of mouse STING shown in first lane of the immunoblot, with densitometric analysis (summed intensity of human STING alleles relative to mouse STING) showing mild STING overexpression in transduced cells. FIG. 6F: HEK293T cells were transfected with plasmids encoding EGFP-human STING, FLAG-232R human STING, and COPA (WT or E241K). Cell lysates were analyzed by western blot, demonstrating absence of HAQ STING phosphorylation in the context of mutant COPA as well as reduced phosphorylation of co-expressed 232R STING. FIG. 6G: Immunoprecipitation of 232R STING was performed in dual human STING containing MEFs, demonstrating co-immunoprecipitation of 232R (major allele) with 232R, 232H, and HAQ alleles. Representative immunoblots of three independent experiments shown.

FIGS. 7A-7C show patient data from derived primary lung fibroblasts. FIG. 7A: Interferon scores calculated from the median fold change in relative quantification values for six ISGs (IFI27, IFI44L, IFIT1, ISG1S, RSAD2, SIGLEC1)(Rice et al., 2013) in primary lung fibroblasts demonstrate decreased interferon activity following introduction of HAQ but not 232R STING. Given high variability between individuals, relative expression normalized to GAPDH was determined for each subject in relation to the mean expression of unmanipulated parental fibroblasts. Statistical testing was performed via 2-way ANOVA within patient cells only, with STING allele significantly affecting interferon scores (p=0.034 for 232R versus HAQ). Šidák's multiple comparison testing was significant for COPA 2 and almost significant for COPA 1 (p=0.087). Bars represent means and error bars represent standard error of the mean. #indicates p<0.1, ** indicates p<0.01. FIG. 7B: STING1 relative expression was normalized within each donor to the mean expression of unmanipulated parental fibroblasts, demonstrating no significant change in STING expression in transduced patient derived cells. Bars reflect mean and error bars standard error of the mean. Expression of 232R versus HAQ STING within a given donor was not statistically different for any individual (repeated measures 2-way ANOVA). STING1 expression in patient cells did not statistically differ from 1 (one sample t-tests). FIG. 7C: Confocal microscopy of control or patient derived fibroblasts transduced with 232R or HAQ STING showed colocalization of 232R but not HAQ STING with cis-Golgi marker GM130 in patients. Intensity histograms across the length of the red indicator are displayed to the right of the images, with STING in green and GM130 in magenta.

FIG. 8 shows interstitial lung disease in COPA syndrome and a pedigree showing the autosomal dominant inheritance of COPA syndrome with variable penetrance.

FIG. 9 shows COPA syndrome patients can develop debilitating arthritis.

FIGS. 10A-10C show interstitial lung disease (ILD) progresses despite immunosuppression and may necessitate lung transplant. FIG. 10A: Age 8; FIG. 10B: Age 13;

FIG. 10C: Age 17.

FIGS. 11A-11F show COPA patients can develop immune-mediated kidney disease necessitating renal transplant.

FIG. 12 shows COPA mediates Golgi to endoplasmic reticulum (ER) transport.

FIG. 13 shows four missense COPA mutations in five families found within 14 amino acid residues of WD40 domain.

FIG. 14 shows mutant COPA leads to impaired binding to KKx proteins and ER stress.

FIGS. 15A-15B show COPA syndrome (FIG. 15A) presents similar to another Mendelian disorder called SAVI (FIG. 15B).

FIG. 16 shows a schematic of intracellular Golgi to ER transport pathways.

FIG. 17 shows STING activation induces multiple cellular responses.

FIG. 18 shows cGAS-STING signaling is linked to many diseases.

FIG. 19 shows loss of STING rescues lung disease in Copa^{E241K/+} mice.

FIG. 20 shows Copa^{E241K/E241K} mice are embryonic lethal and develop loss of digital phenotype.

FIG. 21 shows loss of STING rescues embryonic lethality in Copa^{E241K/E241}K mice.

FIG. 22 shows pedigrees demonstrating COPA syndrome is an autosomal dominant disease with incomplete penetrance.

FIG. 23 shows carriers are clinical unaffected.

FIGS. 24A-24B show patients have markedly earlier symptoms onset (FIG. 24A) and reduced survival (FIG. 24B) relative to carriers.

FIGS. 25A-25B show carriers do not have interstitial lung disease.

FIG. 26 shows a schematic demonstrating that detecting interferons in blood is challenging.

FIG. 27 shows carriers do not have evidence of increased interferon (IFN) activity, a molecular hallmark of COPA syndrome.

FIG. 28 shows single nucleotide polymorphisms (SNPs) of human STING are proposed to influence innate immune responses to cyclic dinucleotides.

FIG. 29 shows unaffected carriers have one copy of HAQ STING.

FIG. 30 shows HAQ STING co-segregates perfectly with clinical non-penetrance.

FIG. 31 shows the SNPs of HAQ STING were the only variants shared by unaffected carriers.

FIG. 32 shows R232H STING does not respond to cGAMP stimulation.

FIG. 33 shows R232H STING does not traffic to the Golgi in response to cGAMP.

FIG. 34 shows 232H STING is not protective in COPA syndrome.

FIG. 35 shows STING activation in mutant COPA cells occurs in the absence of cGAS.

FIGS. 36A-36B show HAQ STING is not activated in the presence of mutant COPA.

FIG. 36A: Data from transfected HEK-293T cells. FIG. 36B: Data from CRISPR-edited HEK-293T cells stabling expressing indicated STING allele.

FIGS. 37A-37B show HAQ STING is not activated after COPA knockdown.

FIGS. 38A-38C show HAQ STING does not accumulate on the Golgi with COPA knockdown.

FIG. 39 shows wildtype (WT) STING forming a hetero dimer with HAQ STING.

FIGS. 40A-40B show HAQ STING acts as a dominant negative to dampen interferon (IFN) signaling.

FIGS. 41A-41D show HAQ STING rescues STING activation in COPA syndrome lung fibroblasts.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, ₉₄%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “corresponding to” or “numbered with reference to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, the position of a selected residue (e.g., a histidine) in a STING protein or STING domain corresponds, for example, to the amino acid position 71 of a reference STING protein, when the selected residue occupies the same essential spatial or structural position as the amino acid at position 71 of the reference STING protein. In some embodiments, where a selected protein is aligned for maximum homology with the reference STING protein the position in the aligned selected protein aligning with the amino acid at position 71 is said to correspond to amino acid position 71. Instead of a primary sequence alignment, a three dimensional structural alignment can also be used, e.g., where the structure of the selected protein (e.g., a pdb structure) is aligned for maximum correspondence with the reference STING protein at, for example, position 71, and the overall structures compared. In this case, an amino acid that occupies the same essential position as the amino acid at position 71 in the structural model is said to correspond to the amino acid at position 71.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an 30 expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “specifically (or selectively) binds to” when referring to a protein or peptide, refers to a binding reaction that is determinative of the presence of the protein, often in a heterogeneous population of proteins and other biologics. Thus, under designated immunoassay conditions, the specified proteins bind to a particular protein at least two times the background and more typically more than 10 to 100 times background.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “genetic variant,” in the context of a particular gene, refers a gene with a variant (e.g., non-standard or abnormal) nucleic acid sequence. The gene includes coding and non-coding sequences, such as regulatory regions. Genetic variants include mutations and polymorphic sequences. Thus, the genetic variant may affect the expression or activity of the gene or gene product. The genetic variant may be an insertion of one or more nucleotides, deletion of one or more nucleotides, or a substitution of one or more nucleotides. A single nucleotide polymorphism (SNP) is an example of a genetic variant.

The term “genetic variant STING allele” refers to a STING gene including a genetic variation. Likewise, the term “genetic variant promoter STING” refers to a variation that is specifically in the promoter region of the STING gene or a STING allele. Similarly, “genetic variant regulatory region STING” and “genetic variant intronic STING” localize the variation within the STING gene or allele. Non-limiting examples of a genetic variant STING allele include, rs11554776, rs78233829, and rs7380824. In embodiments, the genetic variant STING allele is a major STING allele. In embodiments, the genetic variant STING allele is rs1131769. In embodiments, the genetic variant STING allele is a minor STING allele. In embodiments, the genetic variant STING allele is rs11554776. In embodiments, the genetic variant STING allele is rs78233829. In embodiments, the genetic variant STING allele is rs7380824. In embodiments, the genetic variant STING allele mediates clinical penetrance of COPA mutations. In embodiments, the genetic variant STING allele is a pathogenic (disease causing) allele. In embodiments, the genetic variant STING allele is a non-pathogenic (non-disease causing or protective) allele.

The term “allele” is used herein according to its ordinary meaning in the biological arts and refers to the different variants of a gene present at a specific location on a chromosome. For example, at one particular genomic location multiple versions of a nucleotide base may be found and therefore two alleles may exist, one that encodes, for example, for a cytosine base and one that codes for example, for a thymine base.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷SC ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the aspects of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Ph, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the aspects of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88. The term “expression” includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In embodiments, the polynucleotide (e.g., gRNA) is 10, 20, 30, 40, 50, 60, 70, 80, 90, 100 or more nucleic acid residues in length. In embodiments, the polynucleotide (e.g., gRNA) is from 10 to 30 nucleic acid residues in length. In embodiments, the polynucleotide (e.g., gRNA) is 20 nucleic acid residues in length. In embodiments, the length of the polynucleotide (e.g., gRNA) can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the polynucleotide (e.g., gRNA) is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the polynucleotide (e.g., gRNA) is from 10 to 15, 10 to 20, 10 to 30, 10 to 40, or 10 to 50 residues in length.

For specific proteins described herein, the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In embodiments, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference or functional fragment or homolog thereof.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. In embodiments, the Cas9 protein has at least 75% sequence identity to the amino acid sequence of the protein identified by the UniProt reference number Q99ZW2. In embodiments, the Cas9 protein has at least 80% sequence identity to the amino acid sequence of the protein identified by the UniProt reference number Q99ZW2. In embodiments, the Cas9 protein has at least 85% sequence identity to the amino acid sequence of the protein identified by the UniProt reference number Q99ZW2. In embodiments, the Cas9 protein has at least 90% sequence identity to the amino acid sequence of the protein identified by the UniProt reference number Q99ZW2. In embodiments, the Cas9 protein has at least 95% sequence identity to the amino acid sequence of the protein identified by the UniProt reference number Q99ZW2.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In embodiments, the Cpf1 protein is substantially identical to the protein identified by the UniProt reference number U2UMQ6 or a variant or homolog having substantial identity thereto. In embodiments, the Cpf1 protein is identical to the protein identified by the UniProt reference number U2UMQ6. In embodiments, the Cpf1 protein has at least 75% sequence identity to the amino acid sequence of the protein identified by the UniProt reference number U2UMQ6. In embodiments, the Cpf1 protein has at least 80% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 85% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 90% sequence identity to the amino acid sequence of the protein identified by the UniProt reference number U2UMQ6. In embodiments, the Cpf1 protein is identical to the protein identified by the UniProt reference number U2UMQ6. In embodiments, the Cpf1 protein has at least 950 sequence identity to the amino acid sequence of the protein identified by the UniProt reference number U2UMQ6.

Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017. Exemplary Cas9 variants are listed in the Table 1 below.

TABLE 1

| Cas9 Variants | PAM domains | References |
|---|---|---|
| Strep pyogenes (Sp) Cas9 | NGG | Hsu et al. 2014 Cell |
| Staph aureus (Sa) Cas9 | NNGRRT or NNGRR | Ran et al. 2015 Nature |
| | NNGGGT, NNGAAT, NNGAGT | |
| | (Zetsche) | |
| SpCas9 VQR mutant | NGAG > NGAT = NGAA > NGAC | Kleinstiver et al. 2015 |
| (D1135V, R1335Q, T1337R) | NGCG | Nature |
| SpCas9 VRER mutant | NGCG | Kleinstiver et al. 2015 |
| (D1135V/G1218R/R1335E/ | | Nature |
| T1337R) | | |
| SpCas9 D1135E | NGG, greater fidelity, less cutting at | Kleinstiver et al. 2015 |
| | NAG and NGA sites | Nature |
| eSpCas9 1.1 mutant | NGG | Slaymaker et al. Science |
| (K848A/K1003A/R1060A) | | 2015 |
| SpCas9 HF1 | NGG | Kleinstiver et al. 2016 |
| (Q695A, Q926A, N497A, | | Nature |
| R661A) | | |
| AsCpf1 | TTTN (5′ of sgRNA) | Zetsche et al. 2015 Cell |
| HypaCas9 (N692A, M694A, | | Chen et al., Nature |
| Q695A, H698A) | | volume 550, pages 407-410 |
| | | (19 Oct. 2017) |

As used herein, a “zinc finger” is a polypeptide structural motif folded around a bound zinc cation. In embodiments, the polypeptide of a zinc finger has a sequence of the form X₃—Cys-X₂-4-Cys-X₁₂-His-X₃-5-His-X₄, wherein X is any amino acid (e.g., X₂₋₄ indicates an oligopeptide 2-4 amino acids in length). There is generally a wide range of sequence variation in the 28-31 amino acids of the known zinc finger polypeptides. Only the two consensus histidine residues and two consensus cysteine residues bound to the central zinc atom are invariant. Of the remaining residues, three to five are highly conserved, while there may be significant variation among the other residues. Despite the wide range of sequence variation in the polypeptide, zinc fingers of this type have a similar three dimensional structure. However, there is a wide range of binding specificities among the different zinc fingers, i.e. different zinc fingers bind double stranded polynucleotides having a wide range of nucleotides sequences. In embodiments, the zinc finger is the C2H2 type. In embodiments, the zinc finger is the CCHC type. In embodiments, the zinc finger is the PHD type. In embodiments, the zinc finger is the RING type.

The term “TALE” or “transcription activator-like effector” refers to artificial restriction enzymes generated by fusing the TAL effector DNA binding domain to a DNA cleavage domain. TALEs enable efficient, programmable, and specific DNA cleavage and represent powerful tools for genome editing in situ. Transcription activator-like effectors (TALEs) can be quickly engineered to bind practically any DNA sequence. The term TALE, as used herein, is broad and includes a monomeric TALE that can cleave double stranded DNA without assistance from another TALE The term TALE is also used to refer to one or both members of a pair of TALEs that are engineered to work together to cleave DNA at the same site. TALEs that work together may be referred to as a left-TALE and a right-TALE, which references the handedness of DNA. TALE are proteins secreted by Xanthomonas bacteria. The DNA binding domain contains a highly conserved 33-34 amino acid sequence with the exception of the 12th and 13th amino acids. These two locations are highly variable (repeat variable diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

The term “BCMA” or “B-cell maturation antigen” as used herein refers to any of the recombinant or naturally-occurring forms of the cell surface receptor B-cell maturation antigen, also known as tumor necrosis factor receptor superfamily member 17 (TNFRSF17), or variants or homologs thereof that maintain BCMA activity (e.g., within at least 50%, 80%, 90%, 95%, 96%, 97%, 98%, 99% or 100% activity compared to BCMA). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BCMA protein. In embodiments, the BCMA protein is substantially identical to the protein identified by UniProt No. Q02223 or a variant or homolog having substantial identity thereto.

The term “transgene” is used herein according to its plain ordinary meaning and refers to a polynucleotide that encodes an exogenous protein.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene) relative to the activity of the nucleic acid sequence (e.g., transcription of a gene) in the absence of the composition (e.g., fusion protein, complex, nucleic acid, vector). In embodiments, inhibition refers to reduction of a disease or symptoms of disease (e.g., a STING-mediated disease). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In embodiments, the inhibition is 1.5-fold, 2-fold, 3-fold, 4-fold, 5-fold, 10-fold, or more in comparison to a control.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. a STING-mediated disease) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “subject,” “patient,” “individual,” etc. are not intended to be limiting and can be generally interchanged. That is, an individual described as a “patient” does not necessarily have a given disease but may be merely seeking medical advice.

As used herein, the terms “pharmaceutically” acceptable is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose will generally refer to the amount of STING gene therapy. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical and depends on the route of administration.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

As used herein, the terms “treat” and “prevent” are not intended to be absolute terms. Treatment can refer to any delay in onset, reduction in the frequency or severity of symptoms, amelioration of symptoms, improvement in patient comfort and/or respiratory function, etc. The effect of treatment can be compared to an individual or pool of individuals not receiving a given treatment, or to the same patient prior to, or after cessation of, treatment.

The term “prevent” refers to a decrease in the occurrence of STING-mediated disease symptoms in a patient. As indicated above, the prevention may be complete (no detectable symptoms) or partial, such that fewer symptoms are observed than would likely occur absent treatment.

The term “therapeutically effective amount,” as used herein, refers to that amount of the therapeutic agent (e.g., the gene therapy agent) sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “diagnosis” refers to a relative probability that a STING-mediated disease is present in the subject. Similarly, the term “prognosis” refers to a relative probability that a certain future outcome may occur in the subject. For example, in the context of the present invention, prognosis can refer to the likelihood that an individual will develop a STING-mediated disease, or the likely severity of the disease (e.g., severity of symptoms, rate of functional decline, survival, etc.). The terms are not intended to be absolute, as will be appreciated by any one of skill in the field of medical diagnostics.

The terms “correlating” and “associated,” in reference to determination of a STING-mediated disease risk factor, refers to comparing the presence or amount of the risk factor (e.g., dysregulation or genetic variation in a STING gene) in an individual to its presence or amount in persons known to suffer from, or known to be at risk of, the STING-mediated disease, or in persons known to be free of STING-mediated disease, and assigning an increased or decreased probability of having/developing the STING-mediated disease to an individual based on the assay result(s).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The terms “lung disease,” “pulmonary disease,” “pulmonary disorder,” etc. are used interchangeably herein. The term is used to broadly refer to lung disorders characterized by difficulty breathing, coughing, airway discomfort and inflammation, increased mucus, and/or pulmonary fibrosis. Examples of lung diseases include lung cancer, cystic fibrosis, asthma, Chronic Obstructive Pulmonary Disease (COPD), bronchitis, emphysema, bronchiectasis, pulmonary edema, pulmonary fibrosis, sarcoidosis, pulmonary hypertension, pneumonia, tuberculosis, Interstitial Pulmonary Fibrosis (IPF), Interstitial Lung Disease (ILD), Acute Interstitial Pneumonia (AlP), Respiratory Bronchiolitis-associated Interstitial Lung Disease (RBILD), Desquamative Interstitial Pneumonia (DIP), Non-Specific Interstitial Pneumonia (NSIP), Idiopathic Interstitial Pneumonia (IIP), Bronchiolitis obliterans, with Organizing Pneumonia (BOOP), restrictive lung disease, or pleurisy.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBMIAnti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

An “effective amount” is an amount sufficient for a compound (e.g., gene therapy agent) to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology ofPharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

### METHODS

The methods provided herein are, inter alia, useful for the treatment of a STING-mediated disease in a coatomer protein subunit alpha (COPA) syndrome patient who carries a genetic variant STING allele. By introducing a gene therapy (i.e., gene therapy agent) targeting the genetic variant STING allele in said COPA syndrome patient, the patient is treated for the STING-mediated disease. A “STING-mediated disease” as provided herein refers to a disease caused by a genetic variant in a STING gene. The genetic variant STING allele may include a mutation, deletion, duplication, or insertion affecting STING function. The genetic variant STING allele provided herein causing a disease, may also be referred to herein as a pathogenic genetic variant. Thus, a STING-mediated disease may be caused by the presence of a pathogenic genetic variant STING allele (e.g., 232R, rs1131769).

The terms “STING” or “STING gene” as provided herein refer to any of the recombinant or naturally-occurring genes or alleles encoding recombinant or naturally-occurring forms of the stimulator of interferon genes (STING), also known as transmembrane protein 173 (TMEM173) and MPYS/MITA/ERIS or variants or homologs thereof with STING activity (e.g., within at least 50%, 80%, 90%, 95%, 96%, 97%, 98%, 99% or 100% activity compared to STING). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring STING protein. In embodiments, the STING gene is substantially identical to the nucleic acid identified by the GenBank reference number NM_198282.4 or a variant or homolog having substantial identity thereto. In embodiments, the STING gene is substantially identical to the nucleic acid identified by the GenBank reference number XM_291127 or a variant or homolog having substantial identity thereto. In embodiments, the STING protein is encoded by an mRNA substantially identical to the nucleic acid identified by the GenBank reference number XM_005268445 or a variant or homolog having substantial identity thereto. In embodiments, the STING protein is encoded by an mRNA substantially identical to the nucleic acid identified by the GenBank reference number XM_291127 or a variant or homolog having substantial identity thereto. In embodiments, the STING protein is substantially identical to the protein identified by the UniProt reference number Q86WV6 or a variant or homolog having substantial identity thereto. In embodiments, the STING protein is substantially identical to the protein identified by the UniProt reference number Q3TBT3 or a variant or homolog having substantial identity thereto.

A “coatomer protein subunit alpha (COPA) syndrome patient” as provided herein refers to a subject having or being at risk of developing COPA syndrome. “Copa syndrome” as referred to herein is a rare immune-mediated disorder characterized by genetic variations (mutations) in the COPA gene that may occur spontaneously as new mutation or may be inherited in an autosomal dominant pattern. COPA symptoms may appear in childhood (i.e., first or second decade of life). Symptoms and severity of COPA disorder may vary between individual subjects. COPA syndrome subjects may present with autoimmune disorders and/or autoinflammatory disorders. An autoimmune disorder as provided herein is a disorder in which the body's adaptive immune system mistakenly attacks healthy tissue. Autoinflammatory syndromes include disorders characterized by recurrent episodes of inflammation due to an abnormality of the innate immune system. In embodiments, COPA syndrome is a rare autosomal-dominant inborn error of immunity defined by childhood onset of interstitial lung disease (ILD), high-titer autoantibodies, and inflammatory arthritis.

A “STING gene therapy” as provided herein refers to gene therapy methodology and compositions commonly known and available in the art that result at least in part in reversing or improving the pathologic phenotype caused by a genetic variant STING allele. Any known gene therapy composition or gene therapy agent may be used for the STING gene therapy as provided herein and includes without limitation targeted substitution/replacement of a pathogenic (disease-associated or disease-causing) STING allele with a non-pathogenic (healthy, not disease-associated or not disease-causing) STING allele; introducing and expressing (overexpressing) a non-pathogenic (healthy) STING allele in a subject expressing a pathogenic (disease-associated or disease-causing) STING allele. In embodiments, the STING gene therapy includes introducing and expressing a HAQ haplotype in the subject. A HAQ haplotype as provided herein refers to a STING allele including R71H (rs11554776), G230A (rs78233829), and R293Q (rs7380824) single nucleotide polymorphisms (SNPs).

Thus, in an aspect is provided, a method of treating a Stimulator of Interferon Genes (STING)-mediated disease in a subject having or being at risk of having coatomer protein subunit alpha (COPA) syndrome, the method including administering a therapeutically effective amount of a STING gene therapy to the subject thereby treating the STING-mediated disease in the subject.

In another aspect is provided, a method of treating a Stimulator of Interferon Genes (STING)-mediated disease in a subject the method including administering a therapeutically effective amount of a STING gene therapy to the subject thereby treating the STING-mediated disease in the subject.

In embodiments, the STING gene therapy includes gene transfer, gene addition, gene replacement, or genome editing. The gene transfer, gene addition, gene replacement, or genome editing may be performed using methods commonly known in the biological arts. For example, gene addition includes overexpression of a STING gene, STING allele or STING SNP (single nucleotide polymorphism) in the subject by administering to the subject a vector encoding the STING gene, STING allele or STING SNP. The vector may be a lentiviral vector. In addition, STING gene therapy may include administering to the subject a vector encoding a CRISPR targeting Cas9 and guide RNA specific for a STING gene, STING allele or STING SNP to allow for editing of the STING gene, STING allele or STING SNP.

In embodiments, the STING gene therapy includes introducing a histidine at a position corresponding to position 71 of STING, an alanine at a position corresponding to position 230 of STING, and a glutamine at a position corresponding to position 293 of STING to the subject. In embodiments, the STING gene therapy includes introducing a recombinant nucleic acid encoding a histidine at a position corresponding to position 71 of STING, an alanine at a position corresponding to position 230 of STING, and a glutamine at a position corresponding to position 293 of STING to the subject. In embodiments, the STING gene therapy includes expressing a recombinant nucleic acid encoding a histidine at a position corresponding to position 71 of STING, an alanine at a position corresponding to position 230 of STING, and a glutamine at a position corresponding to position 293 of STING in the subject. In embodiments, the recombinant nucleic acid forms part of a lentiviral vector.

In embodiments, the STING gene therapy includes expressing a minor allele of STING. In embodiments, the recombinant nucleic acid sequence encodes a minor allele of STING. In embodiments, the minor allele comprises rs11554776, rs78233829 and rs7380824.

In embodiments, the STING gene therapy includes expressing a recombinant nucleic acid sequence encoding rs11554776, rs78233829 and rs7380824 single nucleotide polymorphisms (SNPs) in the subject. In embodiments, the recombinant nucleic acid sequence forms part of a lentiviral vector.

In embodiments, the STING-mediated diseases is a monogenic autoinflammatory syndrome, an autoimmune disease, a neurological disorder, a metabolic disease, an inflammatory disease, a cardiovascular disease or cancer. In embodiments, the STING-mediated diseases is a monogenic autoinflammatory syndrome. In embodiments, the STING-mediated diseases is an autoimmune disease. In embodiments, the STING-mediated diseases is a neurological disorder. In embodiments, the STING-mediated diseases is a metabolic disease. In embodiments, the STING-mediated diseases is an inflammatory disease. In embodiments, the STING-mediated diseases is a cardiovascular disease. In embodiments, the STING-mediated diseases is cancer.

In embodiments, the STING-mediated diseases is Aicardi Goutieres syndrome (AGS) or Familial chilblain lupus. In embodiments, the STING-mediated diseases is Aicardi Goutieres syndrome (AGS). In embodiments, the STING-mediated diseases is Familial chilblain lupus.

In embodiments, the STING-mediated diseases is systemic lupus erythematosus or rheumatoid arthritis. In embodiments, the STING-mediated diseases is systemic lupus erythematosus. In embodiments, the STING-mediated diseases is rheumatoid arthritis.

In embodiments, the STING-mediated diseases is an ischaemic brain injury, Parkinson disease, general neurodegeneration, Huntington disease, amyotrophic lateral sclerosis, frontotemporal dementia, age-dependent macular degeneration or traumatic brain injury. In embodiments, the STING-mediated diseases is an ischaemic brain injury. In embodiments, the STING-mediated diseases is Parkinson disease. In embodiments, the STING-mediated diseases is general neurodegeneration. In embodiments, the STING-mediated diseases is Huntington disease. In embodiments, the STING-mediated diseases is amyotrophic lateral sclerosis. In embodiments, the STING-mediated diseases is frontotemporal dementia. In embodiments, the STING-mediated diseases is age-dependent macular degeneration. In embodiments, the STING-mediated diseases is traumatic brain injury.

In embodiments, the STING-mediated diseases is nonalcoholic steatohepatitis, alcoholic liver disease or acute pancreatitis. In embodiments, the STING-mediated diseases is nonalcoholic steatohepatitis. In embodiments, the STING-mediated diseases is alcoholic liver disease. In embodiments, the STING-mediated diseases is acute pancreatitis.

In embodiments, the STING-mediated diseases is silica-induced fibrosis or sepsis. In embodiments, the STING-mediated diseases is silica-induced fibrosis. In embodiments, the STING-mediated diseases is sepsis.

In embodiments, the STING-mediated diseases is myocardial infarction or chronic heart failure. In embodiments, the STING-mediated diseases is myocardial infarction. In embodiments, the STING-mediated diseases is chronic heart failure.

In embodiments, the STING-mediated diseases is colorectal cancer, skin cancer or metastases. In embodiments, the STING-mediated diseases is colorectal cancer. In embodiments, the STING-mediated diseases is skin cancer. In embodiments, the STING-mediated diseases is metastases.

In embodiments, the subject is a senescent subject. A senescent subject as provided herein refers to a healthy subject older than 45 years of age or a subject younger than 45 year of age suffering from early onset of aging caused by a disease or genetic disorder.

In embodiments, the subject is not a STING-associated vasculopathy (SAVI) subject. “SAVI” as provided herein is a rare, inherited autoinflammatory disorder characterized by severe inflammation in multiple organs, particularly the skin, blood vessels, and lungs. “Not a STING-associated vasculopathy (SAVI)” is a SAVI that is not caused by genetic variants (e.g., mutations) in the STING gene.

The methods provided herein including embodiments thereof may include steps of diagnosing or prognosing, which include detecting variants of a STING allele in a subject prior to administering the STING gene therapy. The methods may further include a step of detecting a genetic variant STING allele after administering the STING gene therapy. Thus, in embodiments, the method includes prior to the administering a therapeutically effective amount of a STING gene therapy to the subject, detecting an arginine at a position corresponding to position 232 of STING, a histidine at a position corresponding to position 232, a glycine at a position corresponding to position 230 of STING, an arginine at a position corresponding to position 293 of STING, or an arginine at a position corresponding to position 71 of STING.

In embodiments, the presence of the arginine at a position corresponding to position 232 of STING, the histidine at a position corresponding to position 232, the glycine at a position corresponding to position 230 of STING, the arginine at a position corresponding to position 293 of STING, or the arginine at a position corresponding to position 71 of STING indicates the subject has or is at risk of developing a STING-mediated disease.

In embodiments, the method includes prior to the administering a therapeutically effective amount of a STING gene therapy to the subject, detecting a rs1131769 SNP in the subject. In embodiments, the presence of the rs1131769 SNP indicates the subject has or is at risk of developing a STING-mediated disease.

### Examples

### Example 1: The Common HAQ STING Allele Prevents Clinical Penetrance of COPA Syndrome

### SUMMARY

COPA Syndrome is an autosomal-dominant inborn error of immunity that is completely non-penetrant in roughly 20% of individuals, with no known mediators of protection. Recent studies implicate STING in the pathogenesis of COPA Syndrome. Here we show the common HAQ STING allele mediates complete clinical protection from disease. We sequenced 26 affected COPA syndrome patients and 9 unaffected carriers and found HAQ STING entirely co-segregates with clinical non-penetrance. Exome sequencing revealed the only variants shared by unaffected carriers and absent in patients were the mutations comprising HAQ STING. Experimentally, we found that HAQ STING acts in a dominant fashion to dampen COPA-dependent STING signaling. In addition, expressing HAQ STING in patient cells rescued the molecular phenotype of COPA syndrome. Our study is the first report of a common and well-tolerated allele mediating complete clinical protection from a severe genetic disorder. Our findings redefine the diagnostic criteria for COPA syndrome, expose functional differences among STING alleles with broad scientific and clinical implications, and reveal a potential universal gene therapy approach for patients.

### INTRODUCTION

COPA Syndrome is a rare autosomal-dominant inborn error of immunity defined by childhood onset of interstitial lung disease (ILD), high-titer autoantibodies, and inflammatory arthritis (Watkin et al., 2015). COPA Syndrome has reduced penetrance, with 15-30% of individuals with pathogenic coatomer protein alpha (COPA) mutations completely lacking clinical signs and symptoms of disease (Watkin et al., 2015; Fremond and Nathan, 2021; Simchoni et al., 2023).

Pathogenic COPA mutations impair target protein recognition by coat protein complex I (COPI) vesicles, resulting in failed retrieval of client proteins from the Golgi to the endoplasmic reticulum (ER) (Lepelley et al., 2020; Deng et al., 2020; Steiner et al., 2022; Watkin et al., 2015). Murine models have identified STING as central to COPA Syndrome pathogenesis, with loss of STING rescuing embryonic lethality of homozygous Copa^{E241K/E241K} mice and normalizing inflammation in Copa^{E241K/+} mice (Deng et al., 2020). STING signaling is tightly regulated by trafficking; it is only competent to signal in the Golgi (Jeltema et al., 2023). STING may also be important in human COPA Syndrome, although this has not been directly demonstrated and it remains unknown whether other mis-trafficked proteins contribute to disease.

The human STING gene, STING1, is highly pleomorphic. The major 232R allele (rs1131769) accounts for only 57.9% of sequences in the 1000 Genome Project database (Yi et al., 2013). The most common minor allele is the “HAQ” haplotype, comprising the R71H (rs11554776), G230A (rs78233829), and R293Q (rs7380824) single nucleotide polymorphisms (SNPs), that accounts for²0.4% of sequences (Yi et al., 2013). Additional minor alleles are 232H, with a frequency of 13.7%, “AQ”, or G230A and R293Q, with a frequency of 5.2%, and “Q”, or R293Q, with a frequency of 1.5% (Yi et al., 2013). Prior studies evaluating functional differences between STING alleles have been contradictory, with relative function of 232H and HAQ compared to 232R STING highly variable between reports and in response to various stimuli (Jin et al., 2011; Sivick et al., 2017; Patel et al., 2017; Patel and Jin, 2019). Applicant found that STING mis-trafficking is the main mediator of disease in COPA Syndrome, and that STING1 genotype mediates clinical penetrance of COPA mutations.

### Results

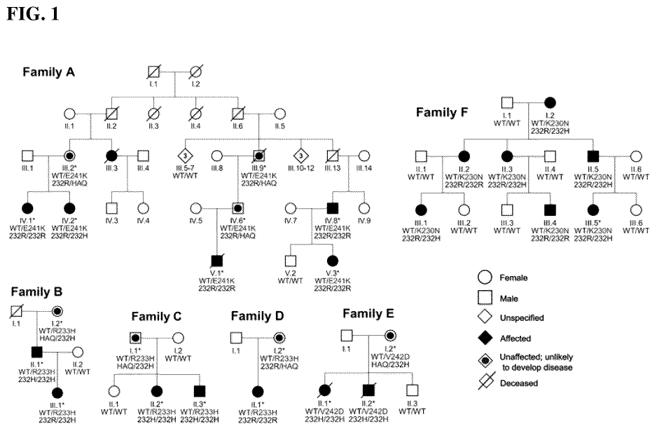

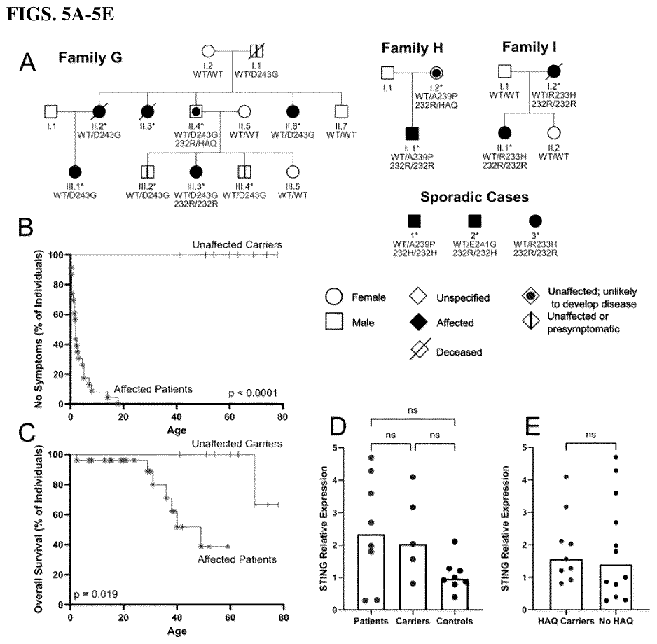

COPA Syndrome patients were identified by clinical or research (Watkin et al., 2015) sequencing and unaffected carriers were identified through familial evaluation (FIG. 1, FIG. 5A). In total, we identified 35 individuals with COPA mutations, including 26 affected patients and 9 unaffected carriers. Five different COPA mutations were seen in affected and unaffected individuals, with 2 further mutations found only in sporadic patients. COPA mutations in study subjects had either been experimentally validated, were alternative amino acids at a validated locus, and/or were present in three or more published case reports.

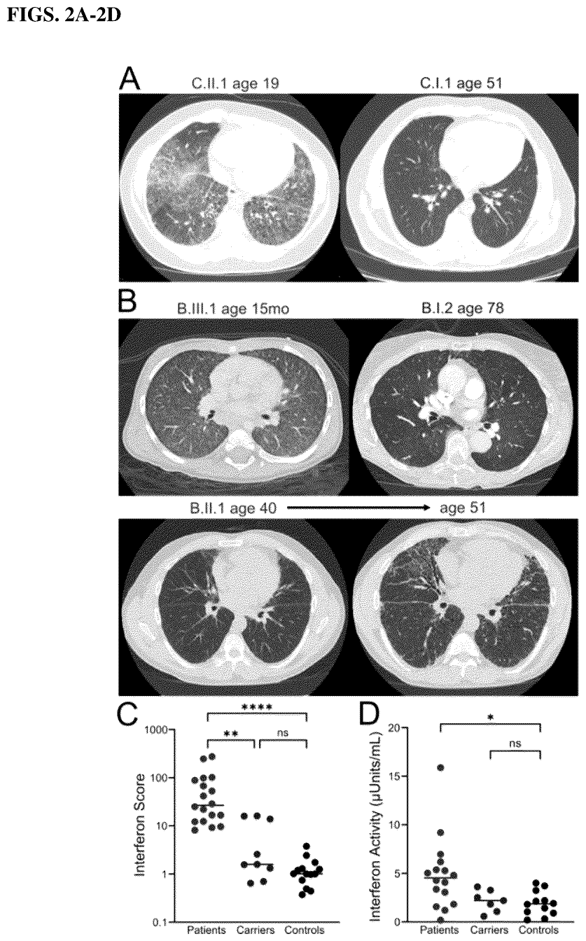

Through medical interviews and detailed chart reviews we found chest imaging abnormalities consistent with COPA Syndrome and high-titer autoantibodies in 100% of patients for whom data were available. These data are consistent with prior studies which found ILD to be universally present in patients with experimentally validated COPA mutations (Watkin et al., 2015; Simchoni et al., 2023; Tsui et al., 2018). None of 9 unaffected carriers had any clinical manifestations compatible with COPA Syndrome including lung, joint, or kidney disease, findings seen in 100%, 82%, and 40%, respectively, of affected patients (Simchoni et al., 2023) and in 100%, 86%, and 47% of patients in this study (Tables 1-2). Unaffected carriers were between 41 and 78 years at most recent clinical evaluation, statistically out of the range of COPA Syndrome presentation of affected patients in this study (Table 1, FIG. 5B, p<0.0001, Gehan-Breslow-Wilcoxon log-rank test). Two of 6 carriers tested, 74- and 78-year-old women, had an elevated antinuclear antibody, a finding seen in up to 20% of healthy older women (Meier et al., 2020). The 74-year-old woman also had a positive rheumatoid factor and positive antineutrophil cytoplasmic antibodies on immunofluorescence, both of which can also be present in healthy adults (Rohm et al., 2024). The 78-year-old woman and four other unaffected carriers of COPA mutations (n=5) underwent chest computed tomography (CT) scans, none of which showed radiographic features of COPA Syndrome, specifically lacking pulmonary cysts, interstitial reticulation, traction bronchiectasis, and diffuse ground glass opacities (GGOs) (FIG. 2A, 2B, Table 2). The 74-year-old unaffected carrier underwent plain chest radiograph examination which also did not show any findings compatible with COPA syndrome. No carriers had signs or symptoms of autoimmune connective tissue disease including arthralgias, myalgias, rashes, or weakness, and none of 7 tested carriers had renal disease (Table 1). Affected patients had increased all-cause mortality relative to unaffected carriers, with a median survival of 49 years (Table 1, FIG. 5C, p=0.019, Gehan-Breslow-Wilcoxon log-rank test). All affected patients passed away from respiratory failure; the deceased unaffected carrier, who had significant tobacco exposure, passed away from bladder cancer.

Having established that unaffected carriers lack clinical manifestations of COPA Syndrome, we evaluated participants on a molecular level, focusing on the type I interferon pathway that is known to be pathologically activated in COPA Syndrome (Kato et al., 2021; Lepelley et al., 2020; Deng et al., 2020). Expanding on prior reports (Lepelley et al., 2020), we found an elevated peripheral blood mononuclear cell (PBMC) interferon score in 12 patients, while 8 unaffected carriers did not differ from controls (FIG. 2C, Table 4). Only affected individuals showed increased serum interferon alpha activity (FIG. 2D, Table 5). Notably, all affected patients were receiving chronic immune suppression, whereas no unaffected carrier received treatment with any of these therapies (Tables 1, 4, 5).

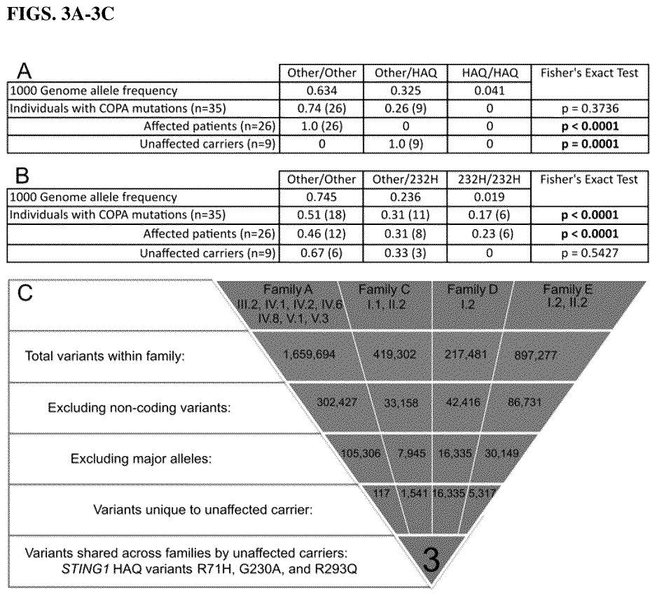

Based on murine models identifying STING as a driver of molecular pathogenesis in COPA Syndrome, we evaluated STING1 genotype from exome (Watkin et al., 2015) (WES) and targeted STING1 sequencing in a total of 35 individuals with COPA mutations, including 26 affected and 9 unaffected individuals. HAQ STING frequency in study subjects matched that seen in the 1000 Genomes Project (Yi et al., 2013), while 232H was more common than expected (FIG. 3A, 3B). Alleles AQ and Q were not seen. Remarkably, a single copy of HAQ STING showed perfect co-segregation with clinical non-penetrance, and no HAQ carriers were seen in a large fully penetrant family (FIG. 3A, 1). In contrast, multiple patients were heterozygous or even homozygous for the 232H allele, indicating this allele is not associated with protection from disease (FIG. 3B).

We next undertook an unbiased search for alternative disease modifying genes via WES analysis of 7 affected patients and 5 unaffected carriers from 4 unrelated kindreds. Sequencing data were filtered to identify variants shared by unaffected individuals, first within and then across families. Remarkably, the only variants shared by all unaffected individuals were R71H, G230A, and R293Q of the HAQ haplotype (FIG. 3C). Similar analysis on a gene level identified only STINGI. Taken together, targeted and unbiased sequencing reveal that a single copy of HAQ STING fully explains clinical non-penetrance, strongly supporting a critical role for STING in human COPA Syndrome pathogenesis as suggested by prior murine data.

Lower STING expression was reported in EBV transformed B cells from HAQ homozygous individuals (Patel et al., 2017), though this was not true in other blood cells (Sivick et al., 2017). We evaluated STINGI expression in PBMCs, finding no differences between controls, unaffected carriers, and patients (FIG. 5D) or between individuals with (all heterozygous) and without the HAQ allele (FIG. 5E).

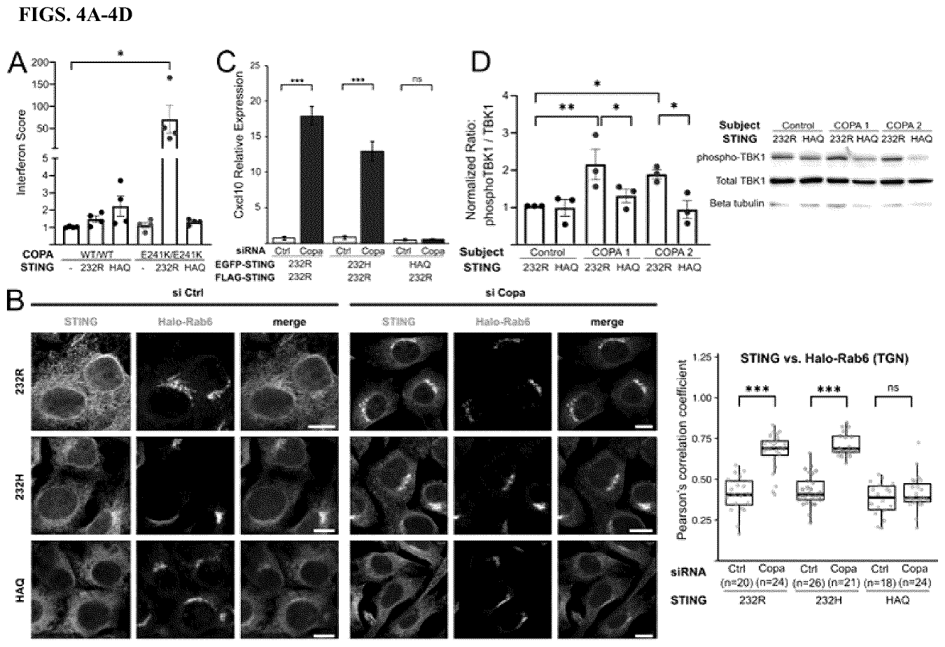

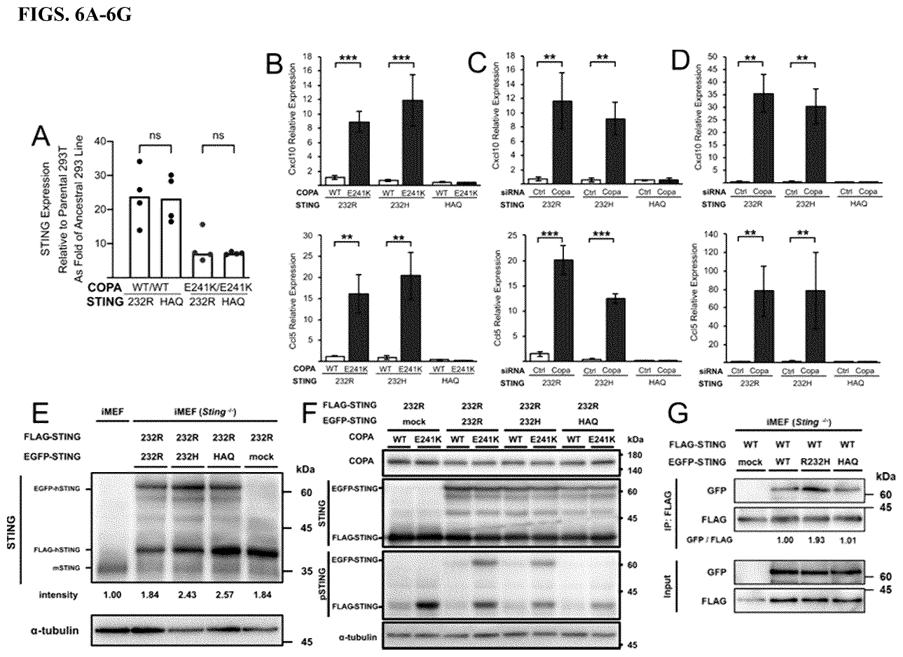

Pathogenic COPA results in ligand-independent accumulation of STING (Deng et al., 2020; Mukai et al., 2021) in the Golgi apparatus and persistent STING signaling at steady state (i.e., homeostatic conditions). To functionally validate our genetic findings, we introduced the E241K COPA mutation into 293T cells, which lack endogenous STING, and generated stable cell lines expressing 232R or HAQ STING at near endogenous levels (FIG. 6A). Mutant COPA cells with 232R STING recapitulated the markedly elevated interferon scores seen in patients, while E241K COPA cells with HAQ STING resembled COPA WT cells (FIG. 4A). Interestingly, prior research has shown that adding the HAQ SNPs onto a constitutively active STING variant abrogated both its pathologic accumulation in the Golgi and interferon signaling (Cerboni et al., 2017). Hence, we speculated that the cellular trafficking of HAQ STING under homeostatic conditions accounted for its ability to resist constitutive activation induced by mutant COPA. Using Sting1−/−mouse embryonic fibroblasts (MEFs), we generated stable cell lines with 232R, 232H, or HAQ human STING. Confocal microscopy of MEFs following COPA depletion demonstrated colocalization of 232R and 232H STING with the Golgi marker Rab6, while HAQ STING localization was not affected by COPA depletion (FIG. 4B). Similar results were seen in MEFs heterozygous for E241K COPA (data not shown).

Golgi localization corresponded with upregulation of inflammatory cytokines for 232R and 232H STING, consistent with prior results in MEFs bearing constitutively active STING (Mukai et al., 2021) (FIGS. 6B and 6C). Consistent with prior reports (Deng et al., 2020; Mukai et al., 2021), cytokine activation induced by 232R and 232H STING in the context of mutant or depleted COPA was independent of the mammalian ligand of STING, cyclic GMP AMP (cGAMP) (FIG. 6D).

A single HAQ STING allele protects unaffected carriers from clinical disease, suggesting that it operates in a dominant fashion. To test this in vitro, we introduced 232R plus 232R, 232H, or HAQ human STING into Sting1−/− MEFs, confirming equal expression of the two STING alleles and total STING expression near endogenous levels (FIG. 6E). Upon COPA depletion, 232R/232R and 232R/232H cells showed increased inflammation, in contrast to 232R/HAQ cells which remained quiescent (FIG. 4C). In addition, co-expression of HAQ STING reduced mutant-COPA dependent phosphorylation of the 232R allele (FIG. 6F). STING obligately forms homodimers (Shang et al., 2019), with cross-allele dimerization a potential mechanism underpinning the ability of HAQ STING to act dominantly. We immunoprecipitated 232R STING in the dual allele MEFs and found similar levels of 232R, 232H, and HAQ STING (FIG. 6G), thereby demonstrating that STING homodimers incorporate alleles stochastically.

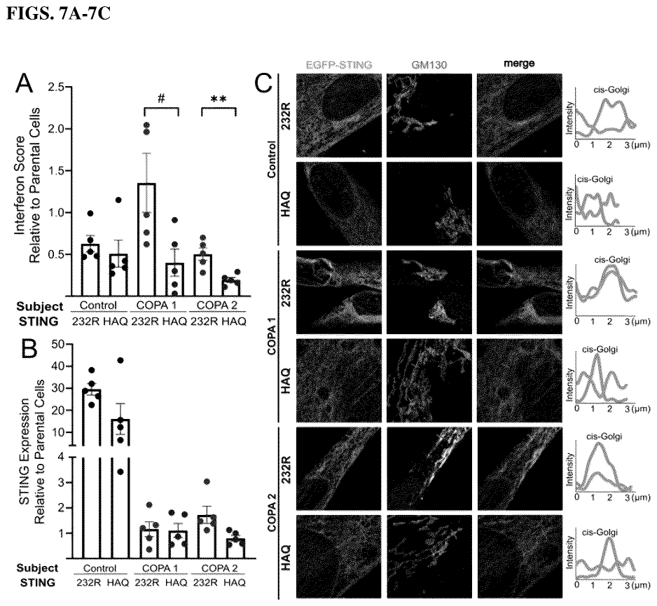

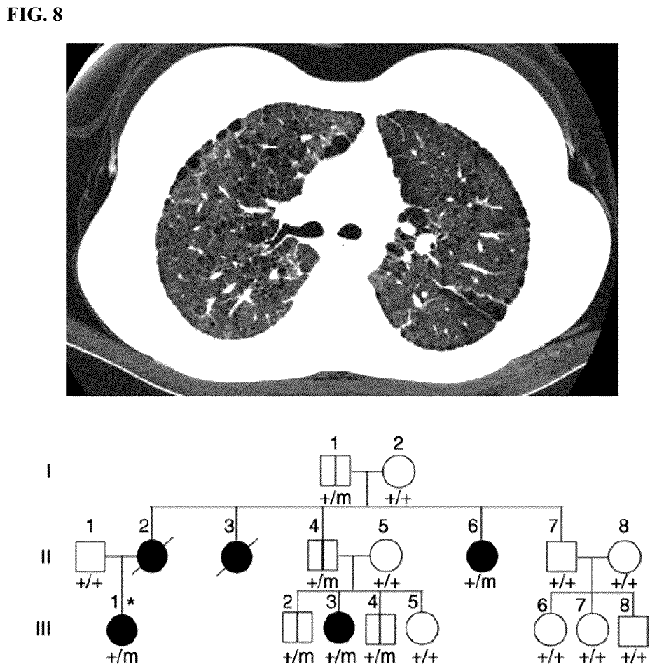

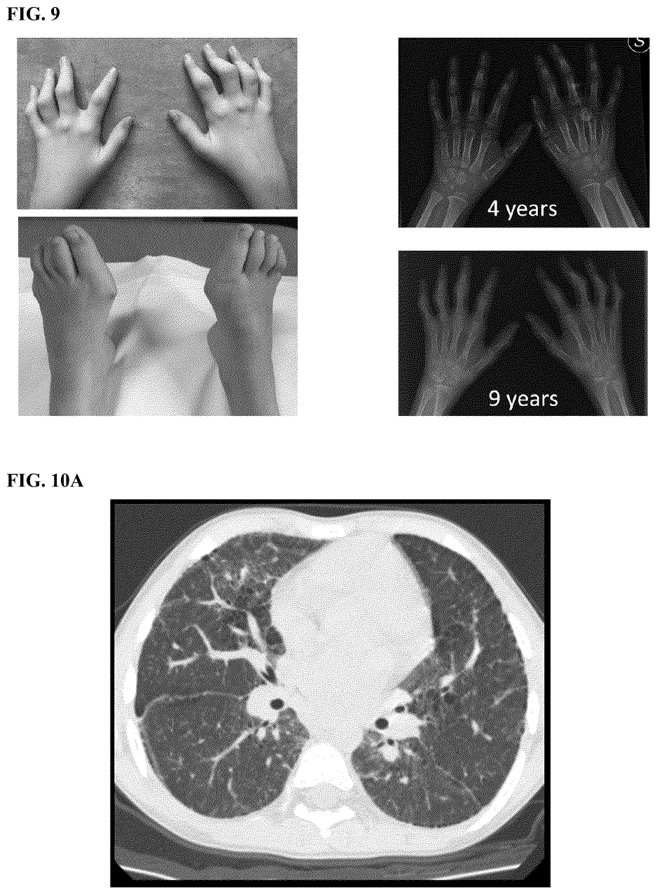

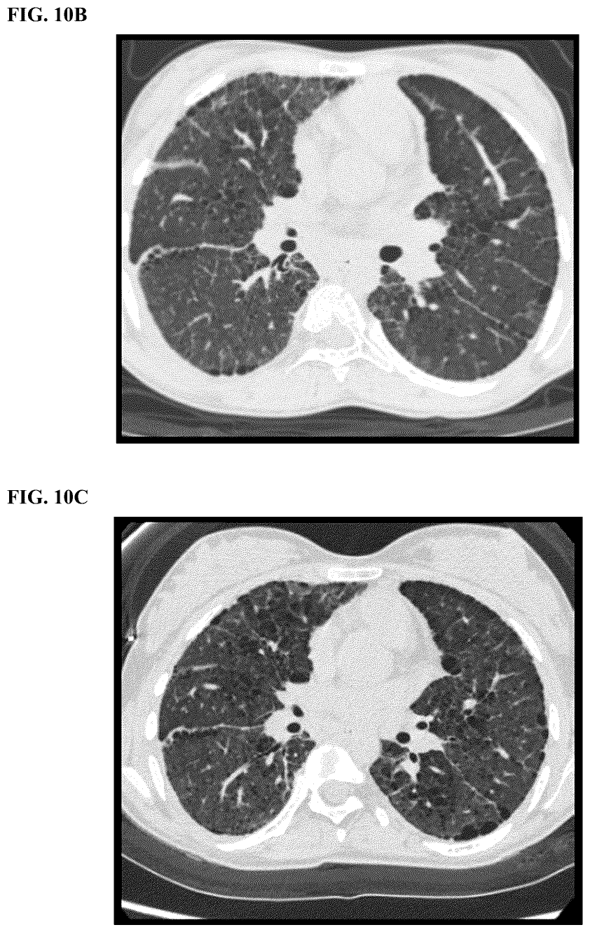

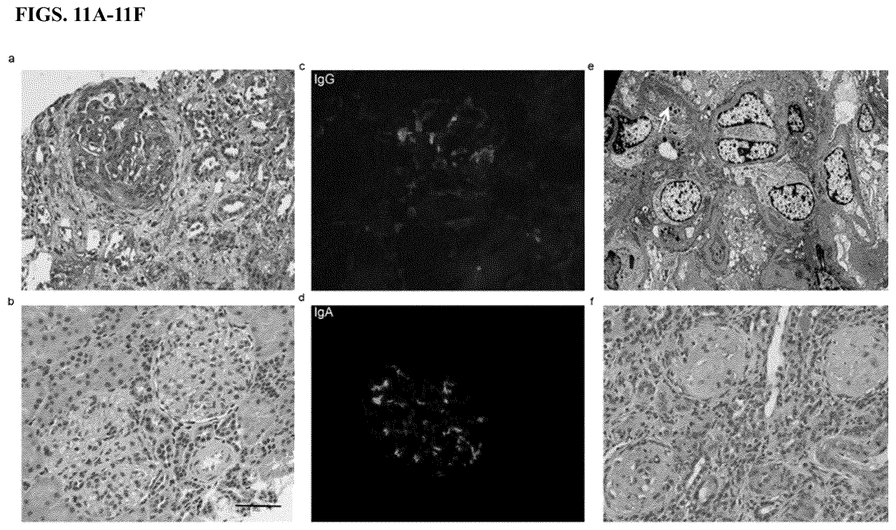

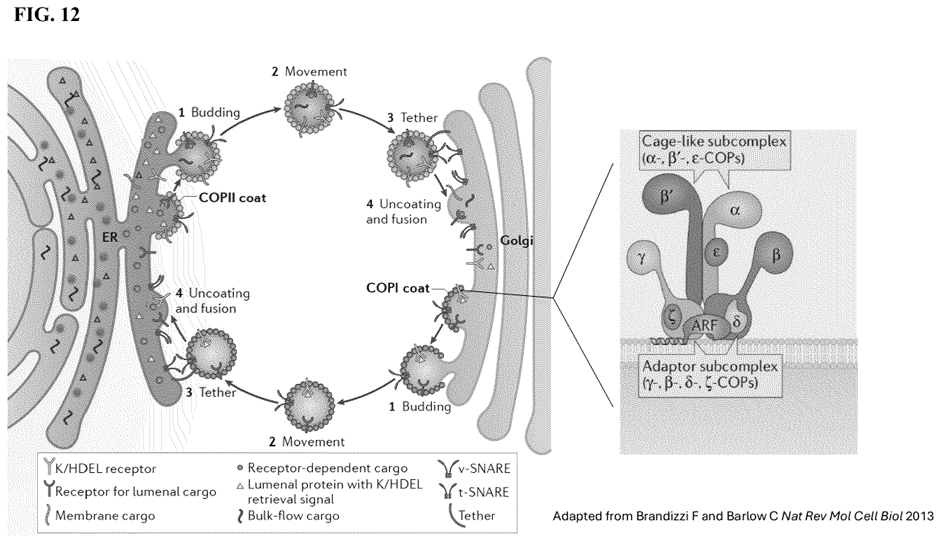

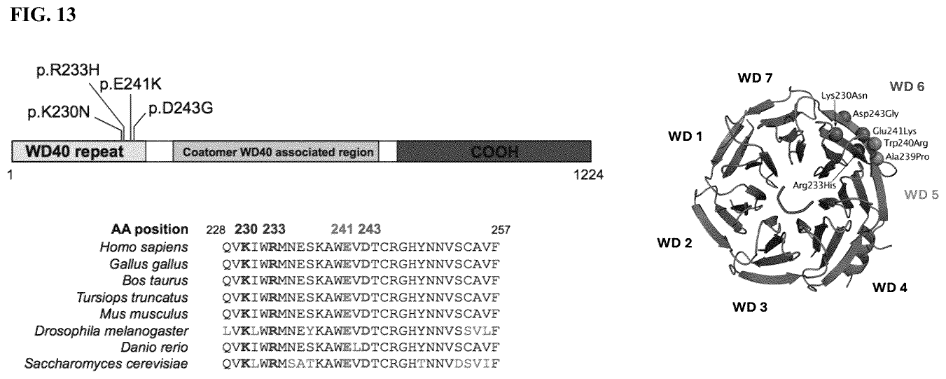

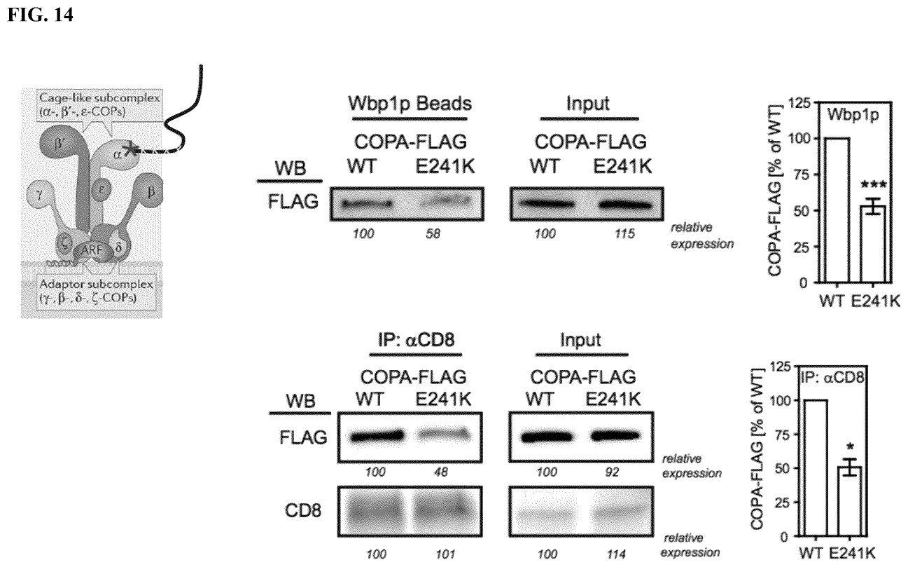

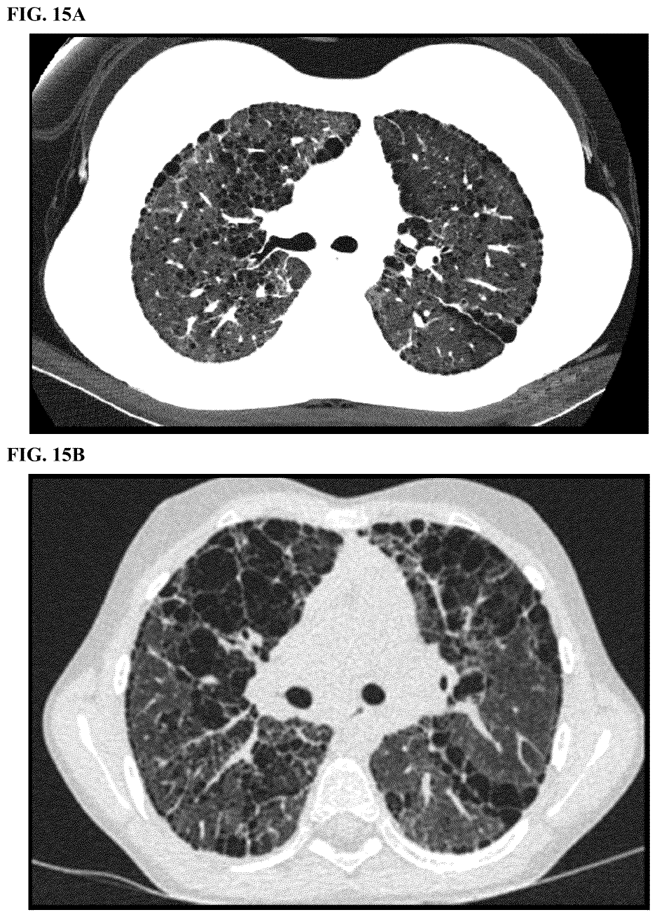

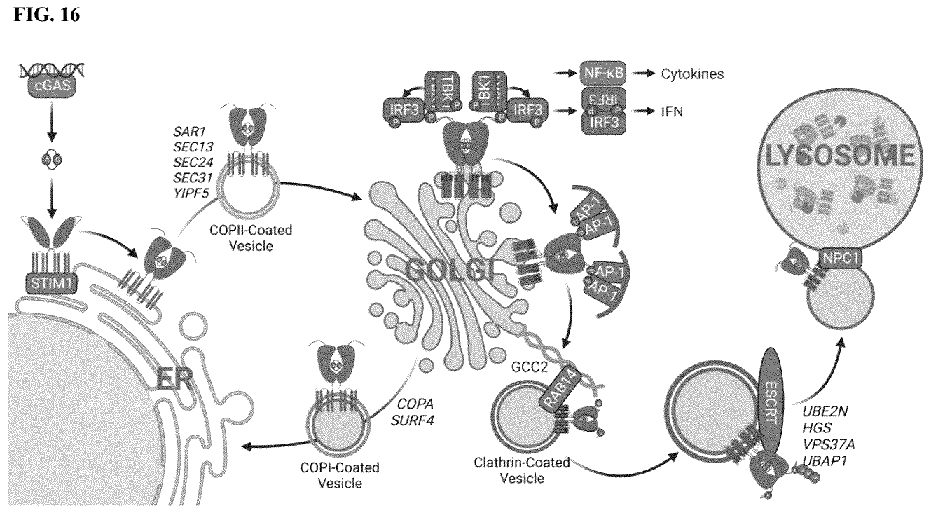

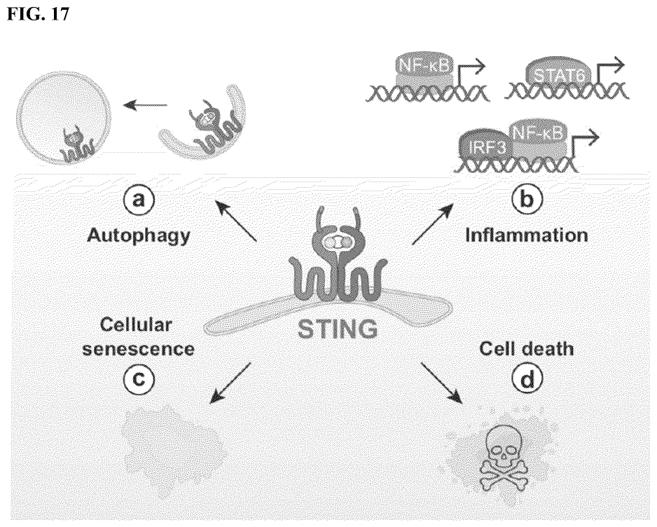

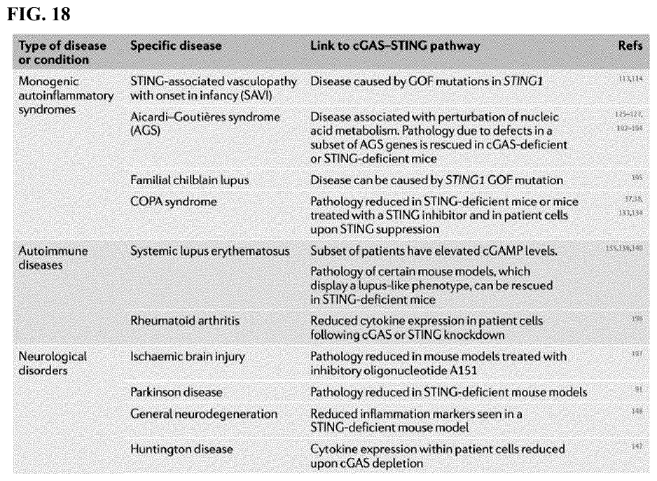

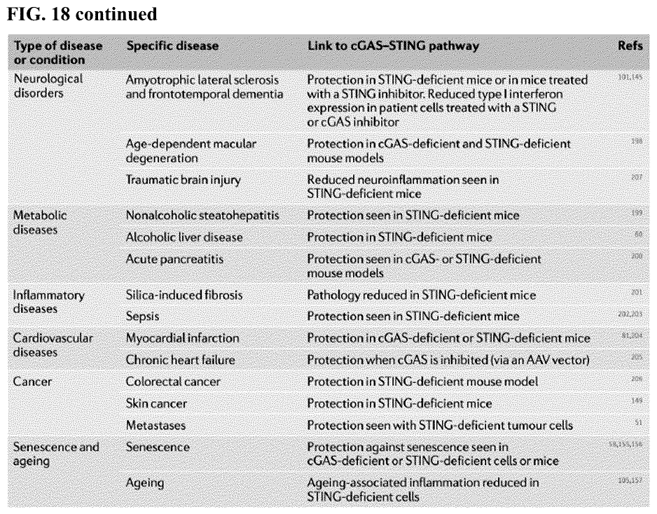

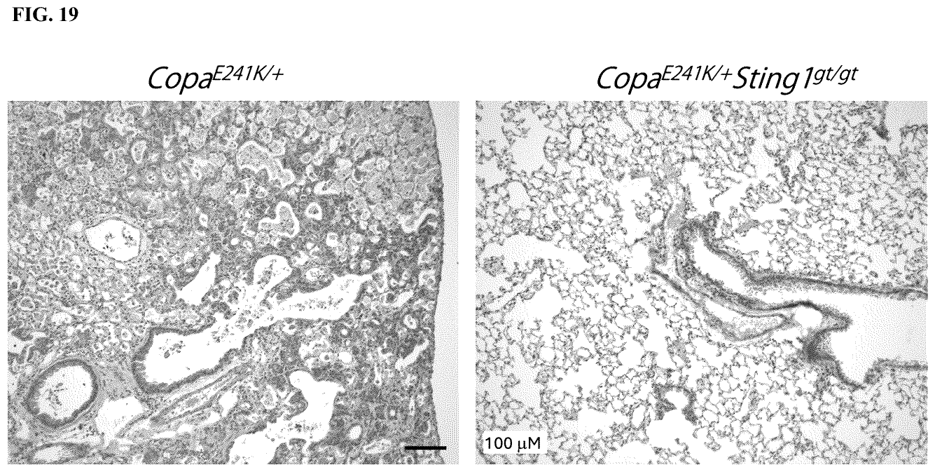

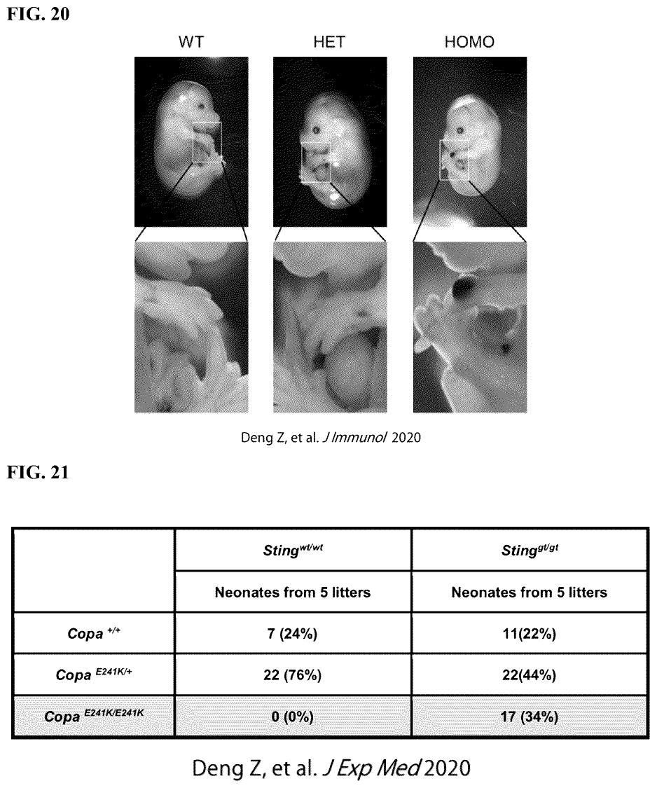

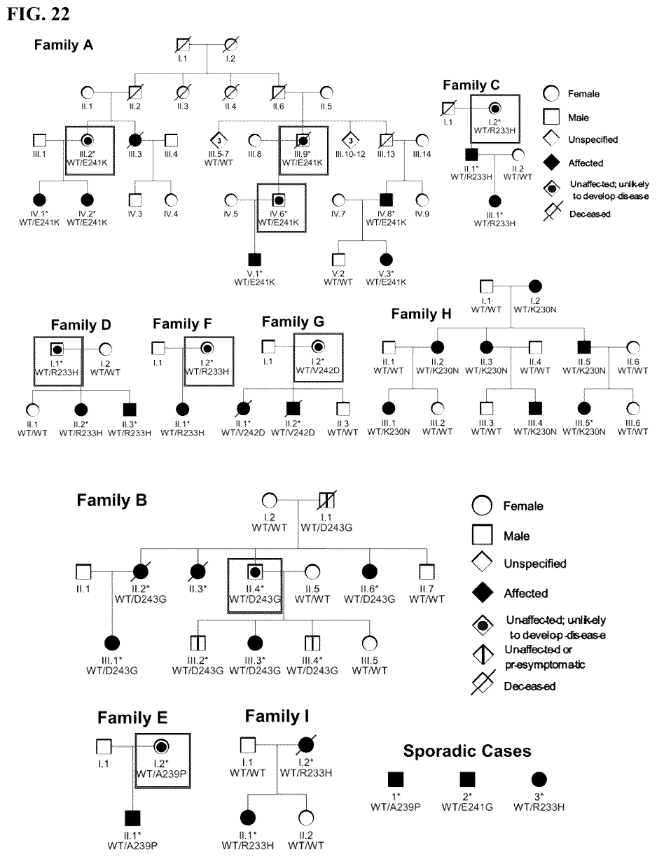

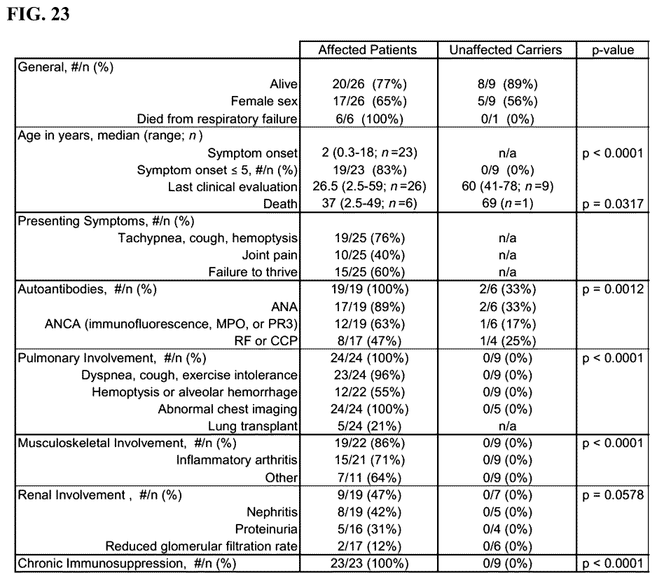

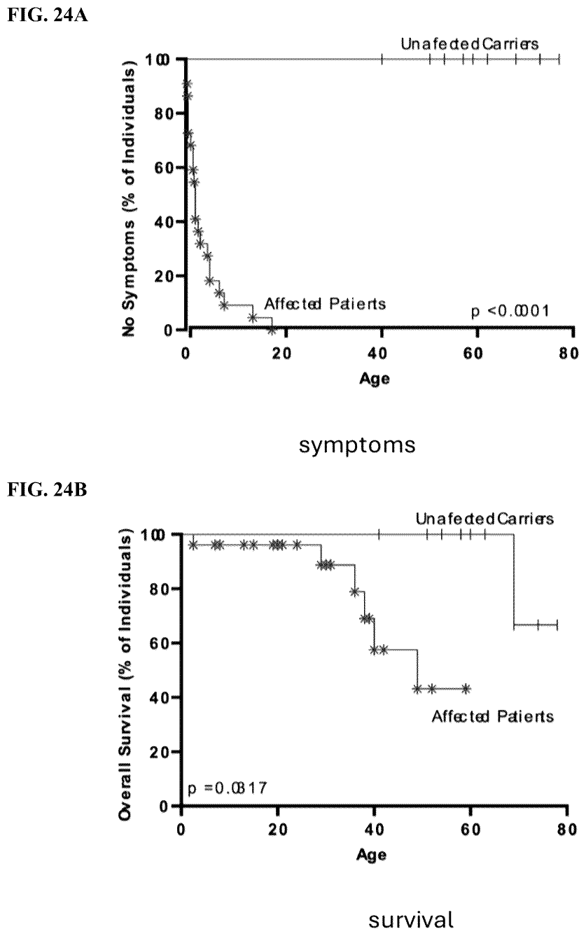

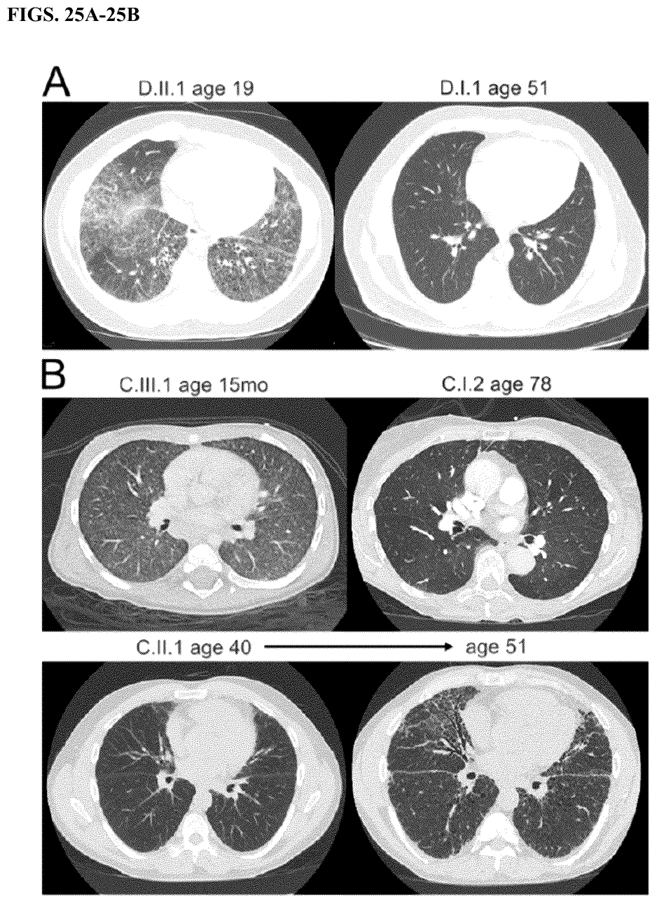

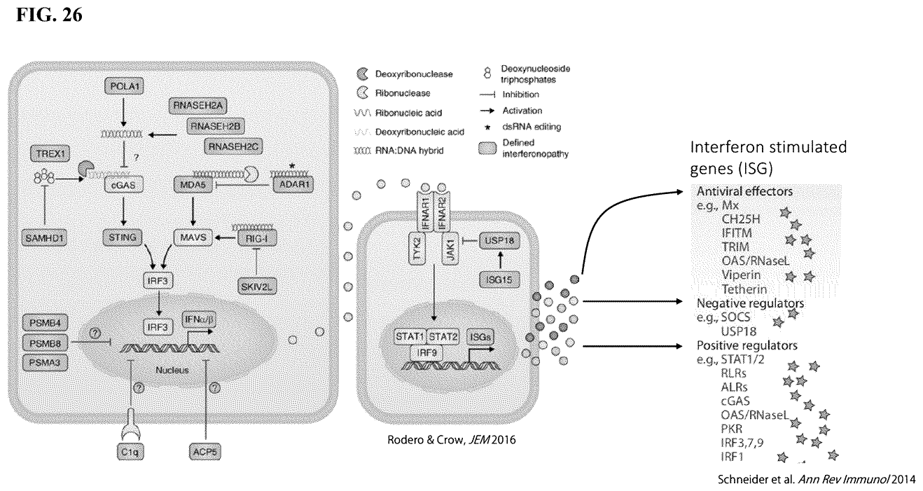

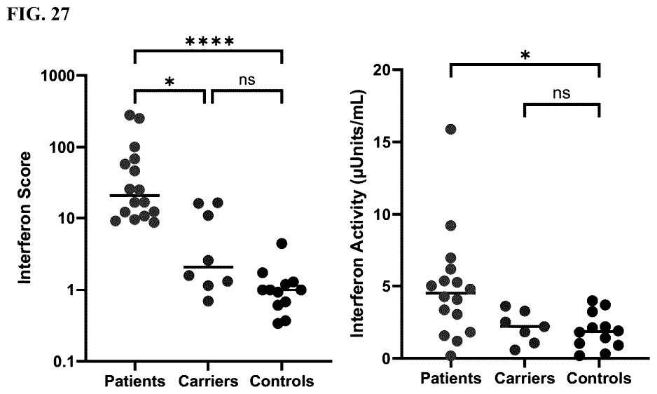

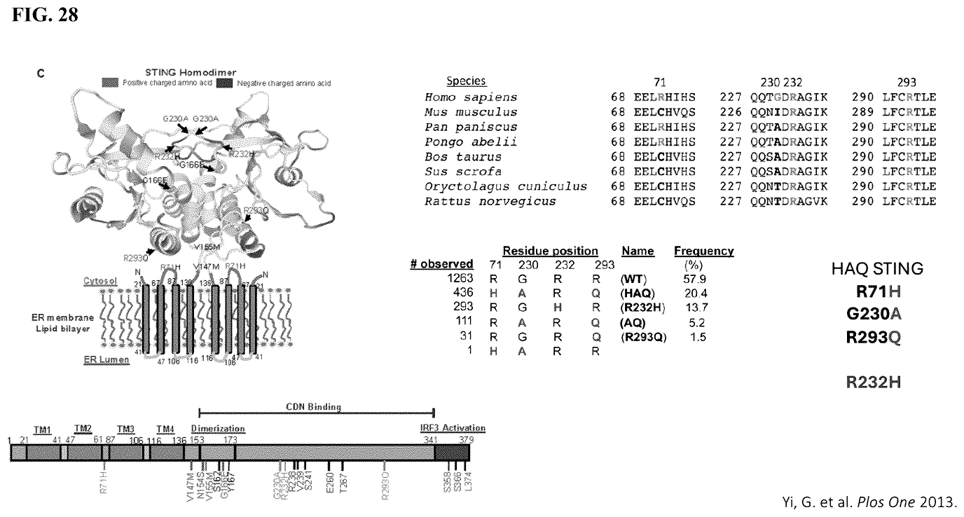

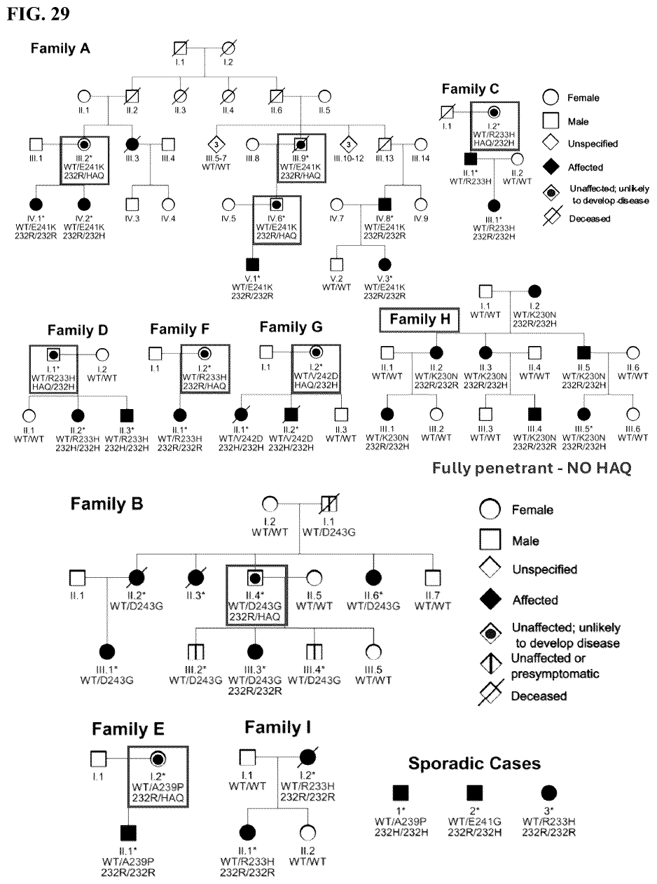

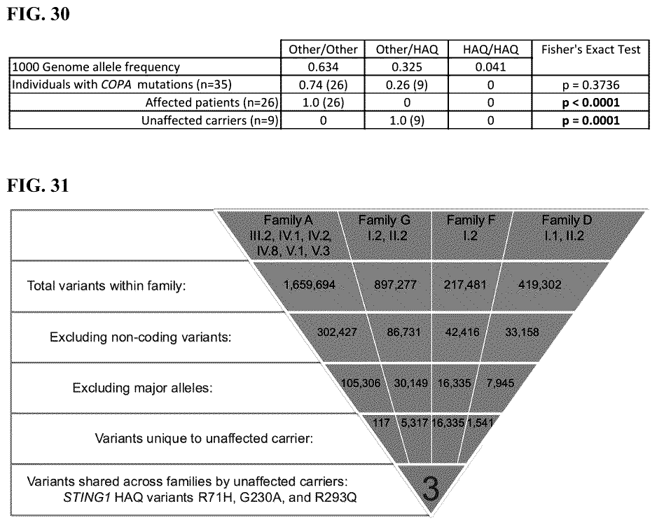

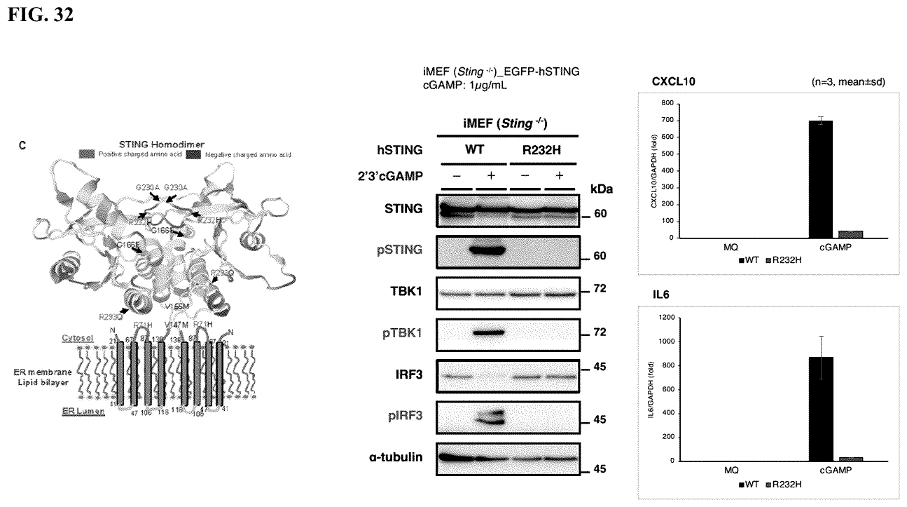

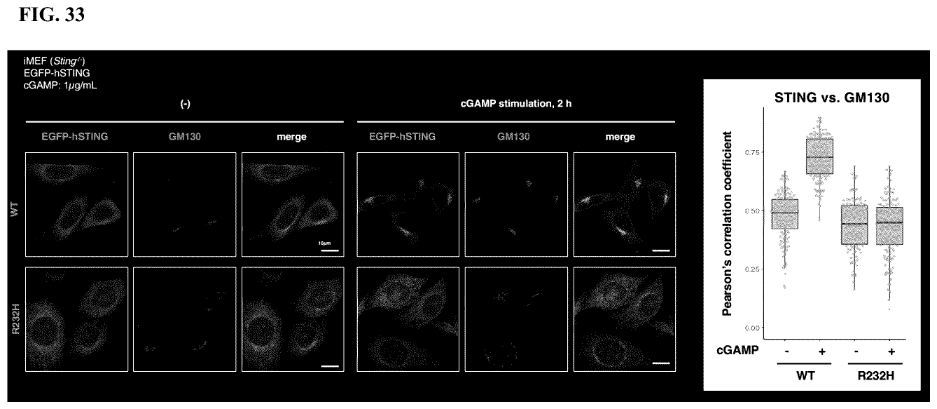

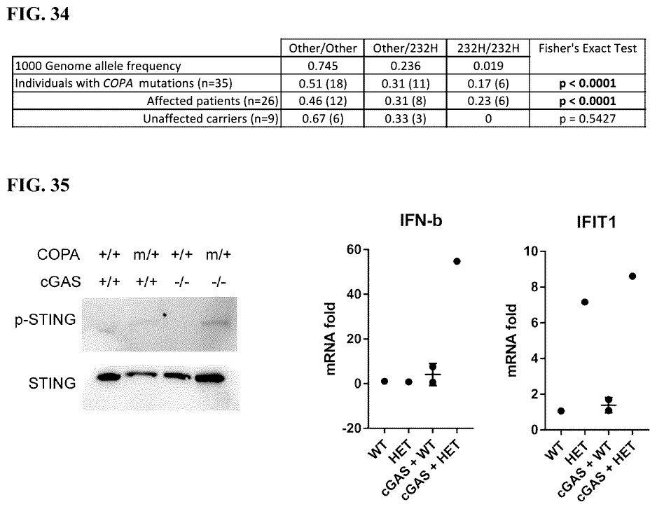

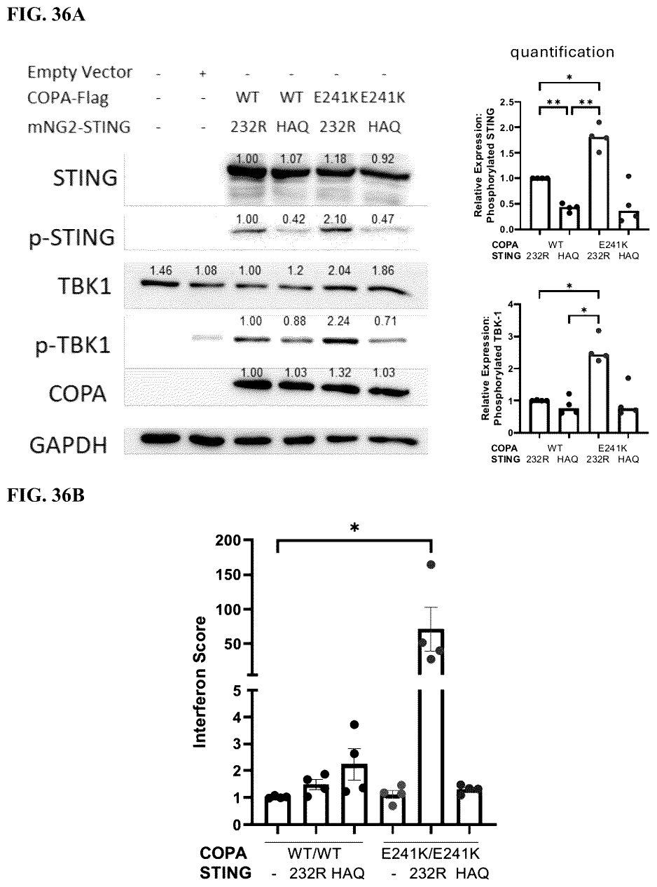

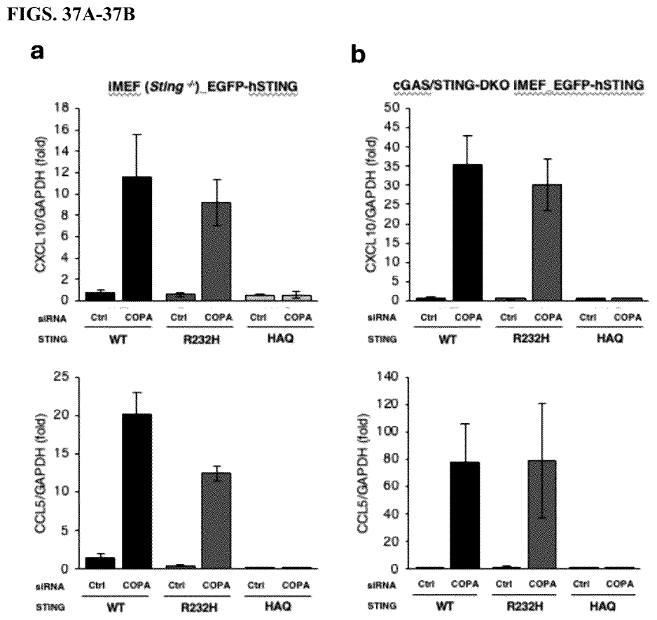

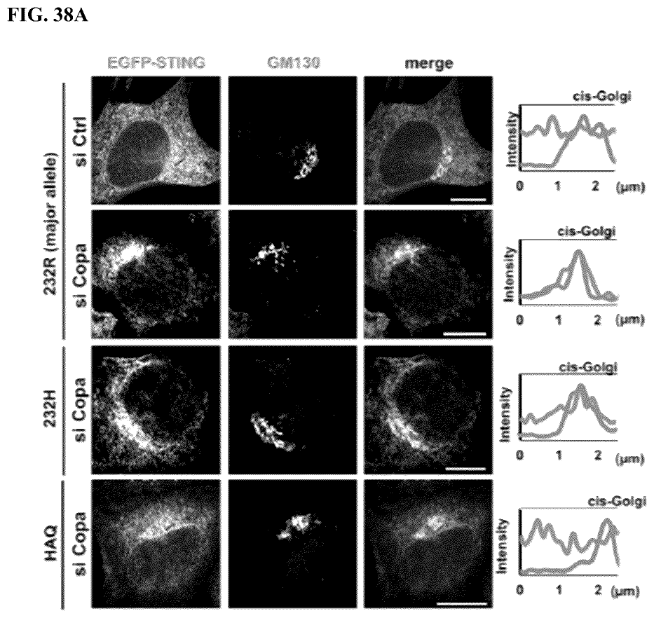

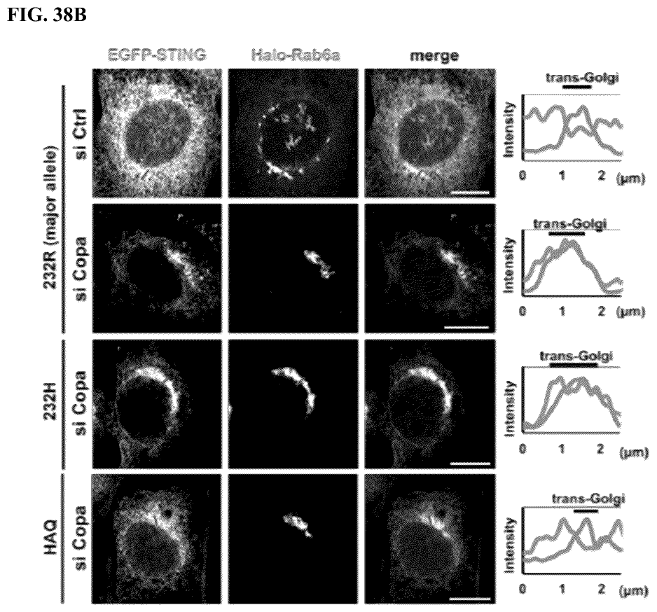

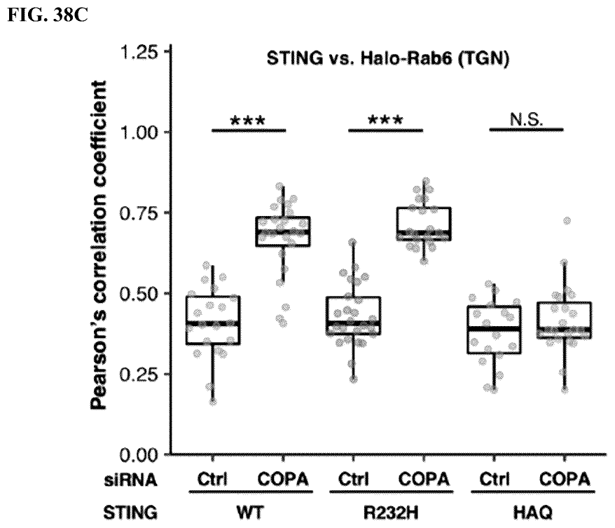

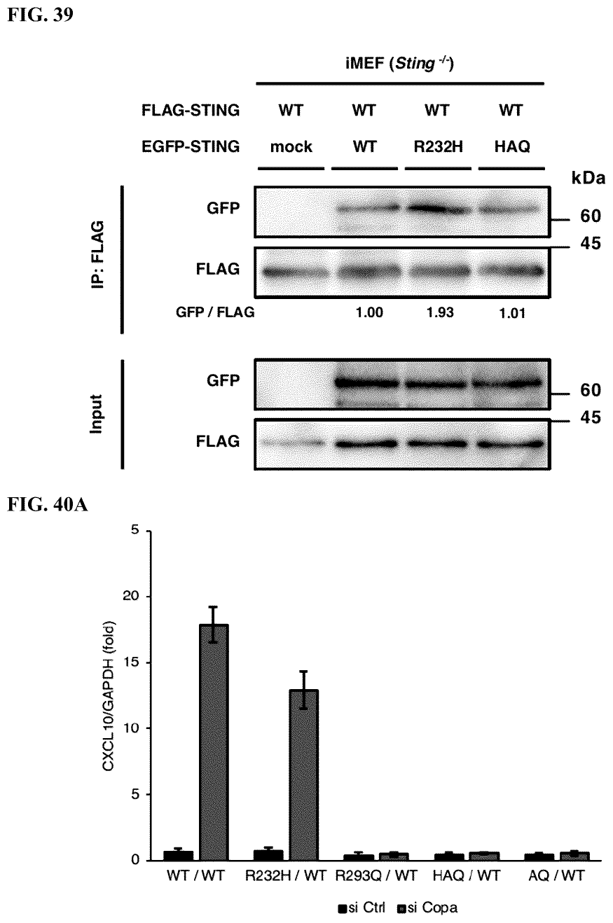

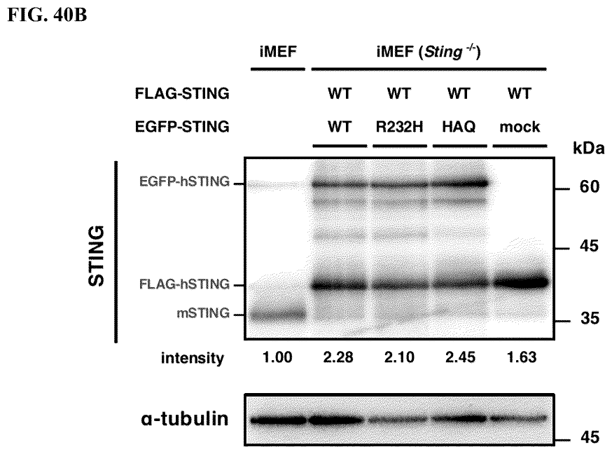

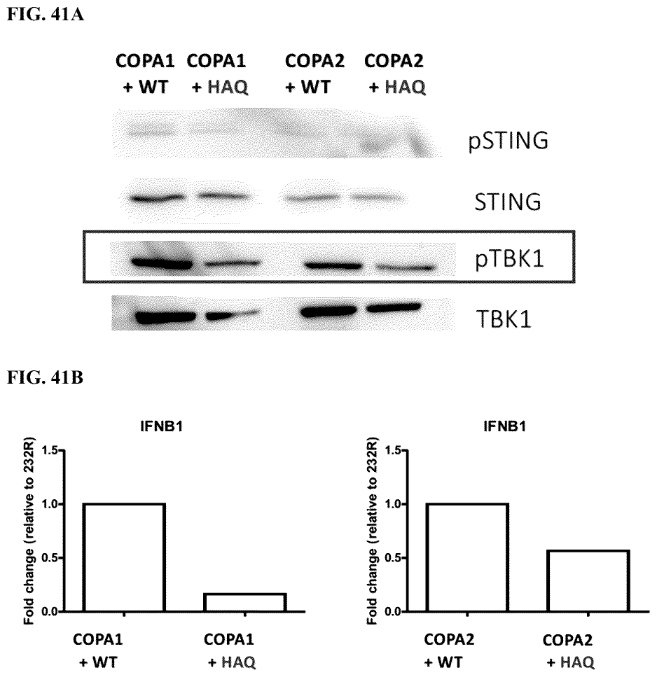

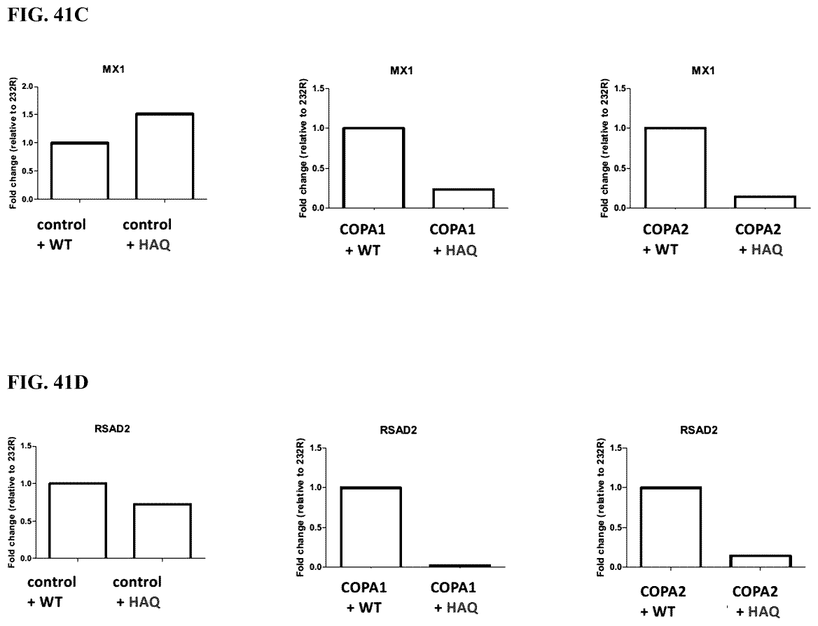

We next evaluated whether introducing HAQ STING into patient cells could correct constitutive STING activation. We examined primary lung fibroblasts from subjects A. V.1 and 1.1.2 that we previously demonstrated to have chronic STING activation (Deng et al., 2020). We transduced cells with 232R or HAQ STING and generated stable cell lines. Activated STING mediates downstream signaling through recruitment and phosphorylation of TBK-1, and, as expected, we saw increased TBK-1 phosphorylation in 232R transduced patient cells while HAQ transduced patient cells resembled control fibroblasts (FIG. 4D). Relative to 232R, HAQ STING also significantly abrogated the interferon signatures in patient cells (FIG. 7A). Importantly, STINGI expression in patient cells remained at endogenous levels seen in unmanipulated fibroblasts (FIG. 7B). Microscopy of transduced STING demonstrated colocalization of 232R STING with cis-Golgi marker GM130 in patient cells, while HAQ STING in patient cells and all STING in control cells were excluded from the Golgi (FIG. 7C). As such, addition of HAQ STING alone into patient cells was sufficient to rescue constitutive STING activation and abrogate downstream interferon signaling.

### DISCUSSION

We show the common HAQ STING allele completely protects individuals with pathogenic COPA mutations from developing clinical disease; the unaffected carriers lack hallmarks of COPA Syndrome including ILD, arthritis, and renal disease. Targeted and unbiased genetic testing revealed that HAQ STING was the only variant shared across unaffected carriers and absent from affected patients, indicating it operates as a genetic suppressor in COPA Syndrome. Our functional studies recapitulated patient findings, with loss of COPA function resulting in STING accumulation in the Golgi, STING activation, and type I interferon induction for the 232R and 232H alleles. None of these findings were seen for HAQ STING; indeed, expression of this allele abrogated chronic STING activation in patient cells. The failure of HAQ STING to undergo constitutive activation despite a defect in COPA function implies that the steady state trafficking of this allele is unique among STING variants.

Our study represents the first report of complete clinical protection from a severe childhood-onset monogenic disease by a common and well tolerated allele. Genetic variants are generally described on a continuum from common, which are expected to have small impacts on health, to strongly impactful rare variants (Claussnitzer et al., 2020; Gruber and Bogunovic, 2020) such as mutations in COPA (Watkin et al., 2015). Genetic modifiers outside the region of a disease-causing gene have been reported in monogenic disorders (Kingdom and Wright, 2022; Rahit and Tarailo-Graovac, 2020; Cooper et al., 2013), yet it is exceedingly rare for such modifiers to completely prevent clinical disease (Arboleda-Velasquez et al., 2019). Identification of suppressor variants powerful enough to impact penetrance can advance the understanding of disease pathology and improve genetic counseling within families. Here we show that COPA Syndrome, which is penetrant in 70-85% (Simchoni et al., 2023) of individuals with pathogenic mutations, is suppressed by the HAQ STING allele carried by 33% of individuals in the 1000 Genome project (Yi et al., 2013). Common alleles, many of which are routinely filtered out during exome and genome analysis, may also impact penetrance of other monogenic disorders (Gruber and Bogunovic, 2020; Kingdom and Wright, 2022).

Our study implicates STING as necessary and sufficient for COPA Syndrome pathogenesis in humans. We believe STING1 genotype should be routinely obtained when evaluating patients for COPA Syndrome as the presence of HAQ would suggest an alternative diagnosis. Our results also support further research into the potential of HAQ STING as gene therapy for COPA Syndrome. This allele is well tolerated by the global population and introducing it may be safer than small molecule STING inhibitors, an alternative therapeutic approach already in clinical development that may increase the risk of infection. Furthermore, genetic addition of HAQ STING would be universal, with no tailoring required for each specific pathogenic COPA mutation. Indeed, absence of ILD in unaffected carriers provides hope that STING based therapies could halt progression of pulmonary fibrosis, the main cause of morbidity and mortality in COPA Syndrome for which no drug class has yet been found to be universally effective (Simchoni et al., 2023).

This study adds to the literature regarding the common 232R, 232H, and HAQ STING alleles. Prior research has been contradictory, reporting alternatively reduced and normal expression levels of STING in individuals homozygous for the HAQ allele (Patel et al., 2017; Sivick et al., 2017). In our study we found similar STING expression levels in PBMCs from individuals heterozygous for or lacking HAQ STING.

Prior functional evaluation of STING alleles has similarly been inconclusive. Different authors have reported 232H and HAQ as both comparable and hypomorphic to 232R in response to stimulation with the mammalian ligand cGAMP or bacterial dinucleotides (Sivick et al., 2017; Patel et al., 2017; Yi et al., 2013). Similar contradictions have been reported in infection models, with HAQ hypomorphic to 232H and 232R in response to Legionella pneumophila (Ruiz-Moreno et al., 2018), while 232H is hypomorphic to 232R and HAQ in response to Herpes simplex (Froechlich et al., 2023).

Unlike these studies, we focused on differences in STING trafficking between the various alleles at steady state rather than in the context of activation by ligands or infections. In the basal flux model of STING biology, homeostatic STING trafficking to the Golgi is balanced by COPI retrieval (Jeltema et al., 2023). In this study, we demonstrated that loss of COPA function impaired retrieval of 232R and 232H STING and triggered activation in the absence of stimulation, in line with multiple prior reports of ligand-independent activation of STING in the context of impaired COPA (Watkin et al., 2015; Lepelley et al., 2020; Steiner et al., 2022; Deng et al., 2020; Mukai et al., 2021; Kemmoku et al., 2024). In stark contrast, HAQ STING did not accumulate in the Golgi or lead to downstream signaling even in the setting of perturbed COPA. This allele may have distinct properties that abrogate the dependence on COPI retrieval seen for other alleles. Alternatively, HAQ STING may have increased affinity for SURF4, the cargo receptor mediating the interaction of COPA and STING (Deng et al., 2020; Mukai et al., 2021; Steiner et al., 2022). Such increased affinity could certainly overcome binding defects of mutated COPA, however it is less clear that this change would overcome COPA depletion.

STING obligately forms homodimers (Shang et al., 2019), which we demonstrated are not allele specific. Cross allele dimerization most likely underlies the ability of HAQ STING to act dominantly.

Understanding the biology of HAQ STING may have further clinical implications as altered trafficking could impact responses to small molecule STING inhibitors and activators under development for cancer (Amouzegar et al., 2021) and autoimmunity (Decout et al., 2021). Allele-specific pre-clinical pharmacodynamics have been reported for some drugs (Pan et al., 2020; Ramanjulu et al., 2018). Our data highlights the importance of studying allele-specific STING responses, suggesting that STING genotyping, which has not been routinely performed (Meric-Bernstam et al., 2022, 2021), should be undertaken as part of future clinical trials.

Limitations of this work include its small sample size, though we note that fewer than 80 individuals with COPA variants have been reported in the literature to date, some of whom have clinical presentations inconsistent with COPA Syndrome (Simchoni et al., 2023). We did not evaluate potential epigenetic contributors to clinical penetrance, nor did we perform a fully comprehensive environmental analysis, though we note that affected and unaffected relatives were seen across the spectrum of urban to rural environments in several countries. We were able to reproduce hallmarks of COPA Syndrome in vitro with complete rescue of both engineered and patient-derived cells by HAQ STING, thus other factors may affect disease severity but are unlikely to explain clinical penetrance. For similar reasons our results argue against an alternative genetic modifier in linkage disequilibrium with the HAQ haplotype.

In summary, we systematically evaluated unaffected carriers of pathogenic COPA mutations to confirm total absence of clinical penetrance. All unaffected carriers carry a single copy of the common HAQ STING allele which afforded them clinical protection. Analysis in vitro recapitulated the inflammatory hallmarks of disease in the presence of COPA perturbation for 232R and 232H but not HAQ STING, and confirmed HAQ STING dominantly mediates protection. Expressing HAQ STING in patient cells abrogated constitutive STING activation, supporting exploration of this approach as possible gene therapy. Future mechanistic studies into altered intracellular trafficking of HAQ STING may further identify the basis of this protection.

### Materials & Methods

### Study Subjects

Subjects were recruited from the pediatric and adult pulmonary and rheumatology clinics at the University of California, San Francisco, Baylor College of Medicine, New York University, The Hospital for Sick Children (Toronto), and The Giannina Gaslini Institute (Genoa) based on diagnosis of COPA Syndrome on clinical or research sequencing. Multiple individuals and families have been previously reported in the literature (Table 3). All study members, or parents for subjects under 18, provided written informed consent to be studied under protocols approved by the Research Ethics Boards or Institutional Review Boards for the protection of human subjects at one of the above institutions. We collected clinical information from subject interviews and medical records.

### Statistical Analysis

Statistical analysis was performed with Prism 10 (GraphPad). Specific statistical tests were used as indicated, including unpaired, non-parametric, two-sided Mann-Whitney test, repeated measures Friedman test with Dunn's multiple comparisons, repeated measures ANOVA with Tukey's multiple comparisons, and repeated measures 2-way ANOVA with Sidak's multiple comparisons test. Frequencies were compared using Fisher's Exact test, and Kaplan-Meier survival analysis was performed using the Gehan-Breslow-Wilcoxon log-rank test. P<0.05 was considered statistically significant.

### Detailed Experimental Methodology

Interferon testing and cytokine gPCR

PBMCs were isolated via Ficoll-Paque gradient density. RNA was extracted from PBMCs (E.Z. N. A Total RNA Kit I, Omega Bio-tek or Quick-RNA MiniPrep, Zymo Research). cDNA was reverse transcribed from 200ng of RNA (SuperScript III, Thermo Fisher Scientific). RT-qPCR analysis was performed with TaqMan Gene Expression Assays from Thermo Fisher Scientific (STING1: Hs00736955_g1; RSAD2: Hs01057264_m1 or Hs00369813_m1; SIGLEC1: Hs00224991_m1; IFI27: Hs01086370_m1 or Hs01086373_g1; IFI44L: Hs00199115_m1 or Hs00915292_m1; IFIT1: Hs00356631_g1 or Hs03027069_s1; ISGJS: Hs00192713_m1 or Hs01921425_s1; STING1 293R/Q genotyping (C_28947918_10)) with expression normalized relative to mean of healthy controls based on expression of GAPDH (Hs02786624_g1) or HPRT1 (Hs03929096_g1) plus 18S (Hs99999901_s1). Interferon score was calculated as median fold expression of six ISGs (Rice et al., 2013). Results for five patients and five controls were previously published (Volpi et al., 2018) while results for all unaffected carriers are novel. Primers used for RT-qPCR of mouse embryonic fibroblasts are listed in Table 6.

Serum interferon alpha activity was assayed by incubating serum isolated using SST tubes (Beckton Dickinson) with the HEK-Blue reporter cell line (Invivogen) prior to determining levels of secreted alkaline phosphatase using QuantiBlue (Invivogen) per manufacturer instructions and read at 620 nm. Standard curve was generated with sequential dilutions of interferon alpha 2b (PBL Assay Science).

### Pedigrees

Pedigree images were created using HaploPainter (Thiele and Nirnberg, 2005).

### Genetic Testing

Genomic DNA was isolated from buccal swabs (Gentra Puregene Buccal Kit, Qiagen), clotted whole blood (Clotspin Baskets and Gentra Puregene Blood Kit, Qiagen), or from purified PBMCs (Gentra Puregene Cells Kit, Qiagen), with all kits used according to manufacturer instructions. STING1 targeted sequencing was performed with previously published primers (Jin et al., 2011), with confirmation of whole exome sequencing results where possible.

### Exome Sequencing and Variant Calling

Exome sequencing for members of Family A was previously reported (Watkin et al., 2015), with identical methodology applied to unaffected carrier D. I.2. Clinical whole exome sequencing was performed for C. I.1 and C. II.2 at Baylor Genetics (Yang et al., 2014). For E. I.2, and E. II.1 whole exome sequencing was conducted on genomic DNA extracted from patient peripheral whole blood through the Agilent SureSelectXT Human All Exon V8 capture kit on Illumina NovaSeqX Plus. For all individuals, the Seq-N-Slide pipeline (igordot.github.io/sns/) was used to perform alignment to the University of California, Santa Cruz hg38 reference genome as well as remove poor quality and duplicate reads. VariantRecalibrator from the Genome Analysis Toolkit (GATK) was used to recalibrate base quality scores and realign INDELS. The following tools from GATK were also used: variant calling was conducted using HaplotypeCaller; individual variant call files were aggregated using genomicsDBImport; GenotypeGVCFs was used for joint genotyping; finally, variants were recalibrated using VQSR. Variants were annotated using VEP (ensembl.org/vep). The variant analysis tool Slivar (github.com/brentp/slivar) was systematically used to narrow down variants of interest. Initially, Slivar's pedigree grouping function was performed to record the total number of variants per family. Next, Slivar restrictions were added to only include coding variants (missense, frameshift, stop gain, nonsense), so called ‘genic’ variants. Further restrictions were implemented to remove variants with population allele frequencies over 0.5 in gnomAD (gnomad.broadinstitute.org/, v. 2.1). Slivar was then used to identify only variants present in unaffected family members within the families analyzed. Finally, only variants shared in unaffected family members between the families were considered.

### Immunoblotting and Antibodies

Cells were lysed in Cold Spring Harbor NP-40 lysis buffer (150 mM NaCl, 50 mM Tris, pH 8.0, and 1.0% Nonidet P-40) containing protease and phosphatase inhibitors (PMSF, NaF, Na₃VO₄, and Roche PhosSTOP). Lysates were cleared by centrifuging at 10,000 g for 10 min at 4° C., size separated on SDS-PAGE gels, and wet transferred onto polyvinylidene fluoride membranes. Membranes were blocked in tris-buffered saline with 0.05% Tween 20 (TBS-T) and 5% nonfat dry milk for 1 h at room temperature, followed by overnight incubation at 4° C. with primary antibodies diluted in TBS-T with 5% BSA. Membranes were washed three times with TBS-T for 10 min, incubated with HRP-conjugated IgG secondary antibody (Jackson Immunoresearch) for 1 h at room temperature, and washed three times with TBS-T for 10 min and once with TBS for 10 min. Lastly bands were visualized with SuperSignal West Femto Chemiluminescent Substrate (Thermo Fisher Scientific) and Bio-Rad's ChemiDoc MP imager.

Primary antibodies were purchased from Cell Signaling Technology: Phospho-STING (D7C3S), Phospho-TBK1 (D52C2), STING (D2P2F), and TBK1 (D1B4); or Santa Cruz Biotechnology: GAPDH (6C5); or Sigma: Flag (F1804).

### Immunoprecipitation

Cells were washed with ice-cold PBS and scraped in immunoprecipitation buffer composed of 50 mM HEPES-NaOH (pH 7.2), 150 mM NaCl, 5 mM EDTA, 1% Triton X-100, protease inhibitor cocktail (25955, dilution) (Nacalai Tesque) and phosphatase inhibitor (8 mM Naf, 12 mM β-glycerophosphate, 1 mM Na3VO4, 1.2 mM Na2MoO4, 5 mM cantharidin, 2 mM imidazole), The cell lysates were centrifuged at 15,000 rpm for 10 min at 4° C., and the resultant supernatants were incubated for 1 h or overnight at 4° C. with anti-DYKDDDDK tag Antibody Beads. The beads were washed three times with immunoprecipitation wash buffer (50 mM HEPES-NaOH (pH 7.2), 150 mM NaCl, 0.1% Triton X-100) and eluted with 2×Laemmli sample buffer. The immunoprecipitated proteins were separated with SDS-PAGE and transferred to the PVDF membrane, then analyzed by western blot.

### Stable Cell Lines

HEK-293T cells were CRISPR edited to introduce the E241K COPA mutation (E241K/E241K); guide RNA and HDR template listed in Table 6. Parental and E241K lines were transduced with lentivirus coding for 232R or HAQ STING fused to N-terminal GFP and maintained under puromycin selection. Polyclonal populations were sorted to obtain single cell clones that were expanded prior to RT-qPCR screening to identify a set of clones with similar STING1 expression (within 2-fold). Clones were kept under puromycin selection. RNA isolation and RT-qPCR analysis was performed as for PBMCs other than cDNA generation with 1 μg of RNA.

Sting1^{-/-} Mouse Embryonic Fibroblasts (MEFs) were generated and transduced with EGFP fused to N-terminal STING variants and HaloTag7 fused to N-terminal Rab6a as previously described (Mukai et al., 2021). MEFs stably expressing EGFP-232R STING were transduced with Flag tag fused to N-terminal STING variants in a similar fashion.

Primary patient fibroblasts were grown from lungs explanted at time of transplantation from subjects A. V.1 (COPA 1) and I. I.1 (COPA 2) as previously described (Deng et al., 2020). Fibroblasts were transduced with EGFP fused to N-terminal STING variants as per MEFs.

### Microscopy

MEFs: cells were fixed with 4% paraformaldehyde (PFA) in PBS at room temperature for 15 min, permeabilized with 0.1% Triton X-100 in PBS at room temperature for 5 min, blocked with 3% BSA in PBS, and incubated with anti-GM130 antibody (BD Biosciences, clone 35). After washing with PBS three times, cells were then incubated with the secondary antibody at room temperature for 60 min, washed, and mounted with ProLong™ Glass Antifade Mountant (P36982, Thermo Fisher Scientific). For staining of Halo-Rab6a, cells were incubated with HaloTag® SaraFluor 650 T Ligand (1 pM) at room temperature for 30 min as previously described (Kuchitsu et al., 2023).

Primary human lung fibroblasts: cells were seeded onto glass coverslips coated with poly-L-lysine hydrobromide (MP Biomedicals) and treated as indicated. Cells were then fixed with 4% paraformaldehyde, permeabilized with 0.1% Triton X-100, and blocked with 3% BSA. Slides were incubated with anti-GM130 antibody (BD Biosciences, clone 35) antibody overnight at 4° C. and incubated with fluorescent-conjugated secondary antibody for 60 min at room temperature. Slides were then mounted with FluorSave Reagent (Millipore) and kept at 4° C. in the dark. Images were captured with a Leica TCS SPE microscope, 63×objective, and oil immersion as previously described (Deng et al., 2020).

### Quantification of Imaging Data

Individual cells were manually segmented to quantify imaging data from multiple cells. Pearson's correlation coefficient was quantified by BIOP JACoP in Fiji plugin with region of interest (ROI) data from Cellpose.

### Tables

TABLE 1
Clinical characteristics of patients and unaffected carriers of COPA mutations.

| | | Unaffected | |
|---|---|---|---|
| | Affected Patients | Carriers | p-value |
| General, #/n (%) | | | |
| Alive | 19/26 (73%) | 8/9 (89%) | |
| Female sex | 17/26 (65%) | 5/9 (56%) | |
| Died from respiratory failure | 7/7 (100%) | 0/1 (0%) | |
| Age in years, median (range; n) | | | |
| Symptom onset | 2 (0.3-18; n = 23) | n/a | p < 0.0001 |
| Symptom onset ≤5, #/n (%) | 19/23 (83%) | 0/9 (0%) | |
| Last clinical evaluation | 26.5 (2.5-59; n = 26) | 60 (41-78; n = 9) | |
| Death | 36 (2.5-49; n = 7) | 69 (69-69; n = 1) | p = 0.019 |
| Presenting symptoms, #/n (%) | | | |
| Tachypnea, cough, hemoptysis | 19/25 (76%) | n/a | |
| Joint pain | 10/25 (40%) | n/a | |
| Failure to thrive | 15/25 (60%) | n/a | |
| Autoantibodies, #/n (%) | 19/19 (100%) | 2/6 (33%) | p = 0.0012 |
| ANA | 17/19 (89%) | 2/6 (33%) | |
| ANCA (immunofluorescence, | 12/19 (63%) | 1/6 (17%) | |
| MPO, or PR3) | | | |
| RF or CCP | 8/17 (47%) | 1/4 (25%) | |
| Pulmonary involvement, #/n | 24/24 (100%) | 0/9 (0%) | p < 0.0001 |
| (%) | | | |
| Dyspnea, cough, exercise | 23/24 (96%) | 0/9 (0%) | |
| intolerance | | | |
| Hemoptysis or alveolar | 12/22 (55%) | 0/9 (0%) | |
| hemorrhage | | | |
| COPA Syndrome features on | 23/23 (100%) | 0/6 (0%) | |
| chest imaging | | | |
| Lung transplant | 5/24 (21%) | n/a | |
| Musculoskeletal involvement, | 19/22 (86%) | 0/9 (0%) | p < 0.0001 |
| #/n (%) | | | |
| Inflammatory arthritis | 15/21 (71%) | 0/9 (0%) | |
| Other | 7/11 (64%) | 0/9 (0%) | |
| Renal involvement, #/n (%) | 9/19 (47%) | 0/7 (0%) | p = 0.0578 |
| Nephritis | 8/19 (42%) | 0/5 (0%) | |
| Proteinuria | 5/16 (31%) | 0/4 (0%) | |
| Reduced glomerular filtration | 2/17 (12%) | 0/7 (0%) | |
| rate | | | |
| Chronic immunosuppression, | 23/23 (100%) | 0/9 (0%) | p < 0.0001 |
| #/n (%) | | | |

Footnotes: Chest imaging refers to computed tomography (CT) scans except for one unaffected carrier with plain a chest radiograph. Autoantibody titer of 1:80 or higher considered positive for ANA. Lung transplants include one patient with combined heart-lung transplant and one patient with re-do lung transplantation. Nephritis defined by hematuria, excluding known nephrolithiasis, or diagnosis on biopsy. Other musculoskeletal involvement includes arthralgias, myalgias, avascular necrosis, or need for orthopedic surgery except when indication was trauma. Proteinuria defined as 2 or more instances of urine protein creatinine ratio (UPCR) >1. Reduced glomerular filtration rate defined as ≤60. Statistical analysis performed by log-rank test for ages at symptom onset and death or by Fisher Exact test for various disease manifestations.
Abbreviations: ANA = antinuclear antibody; ANCA = antineutrophil cytoplasmic antibody; MPO = myeloperoxidase antibody; PR3 = proteinase 3 antibody; RF = Rheumatoid Factor; CCP = anti- cyclic citrullinated peptide antibody.

TABLE 2
Detailed demographic and clinical information of select patients and unaffected carriers.

| Identifier | | A.IV.1 | A.V.1 | A.III.2 | A.III.9 | A.IV.6 |
|---|---|---|---|---|---|---|
| General | COPA | WT/E241K | WT/E241K | WT/E241K | WT/E241K | WT/E241K |
| | genotype | | | | | |
| | STING | 232R/232R | 232R/232R | 232R/HAQ | 232R/HAQ | 232R/HAQ |
| | genotype | | | | | |
| | Race, | white, non- | white, non- | white, non- | white, non- | white, non- |
| | ethnicity | Hispanic | Hispanic | Hispanic | Hispanic | Hispanic |
| | Status | alive | deceased | alive | deceased | alive |
| | Cause of | | lung disease | | bladder cancer | |
| | Death | | | | | |
| | Sex | female | male | female | male | male |
| Age | Symptom | 2 years | 3 months | n/a | n/a | n/a |
| | onset | | | | | |
| | Last | 42 years | 31 years | 63 years | 69 years | 54 years |
| | evaluation | | | | | |
| | Death | | 31 years | | 69 years | |
| Presenting | Pulmonary | | + | | | |
| symptoms | Joint | + | | | | |
| | FTT | | + | | | |
| Autoantibodies | ANA | positive, | 1:320, speckled | negative | n/a | n/a |
| | | speckled | or homogeneous | | | |
| | ANCA/MPO/PR3 | n/a; 19-46 | pANCA; neg; neg | n/a; neg; neg | n/a; n/a; n/a | n/a; n/a; n/a |
| | | EU/mL; neg | | | | |
| | RF/CCP | neg; neg | neg; n/a | neg; neg | n/a; n/a | n/a; n/a |
| Pulmonary | Symptoms | yes | yes | none | none | none |
| | Hemorrhage | yes | no | no | no | no |
| | CT consistent | yes | yes | no | n/a | no |
| | with COPA | supportive | supportive | reticulation, | | no GGOs, |
| | Syndrome | findings: | findings: | traction | | reticulation, |
| | | reticulations, | discrete | bronchiectasis, or | | cystic |
| | | fibrosis, | cystic changes, | cystic changes | | changes, |
| | | honeycombing, | reticulations, | | | traction |
| | | cystic changes | GGOs | | | bronchiectasis; |
| | | | | | | +smoking related |
| | | | | | | emphysema |
| | Transplant | undergoing | lung | not indicated | not indicated | not indicated |
| | | evaluation | transplant, | | | |
| | | | retransplant | | | |
| | | | after 5 years | | | |
| | Biopsy | follicular | follicular | not indicated | not indicated | not indicated |
| | | bronchiolitis, | bronchiolitis | | | |
| | | interstitial | with | | | |
| | | fibrosis, | interstitial | | | |
| | | alveolar | inflammation | | | |
| | | hemosiderosis | and fibrosis | | | |
| | | (20 y) | (5 mo & 2 y) | | | |
| MSK | Arthritis | polyarthritis | no | no | no | no |
| Involvement | Other | bilateral | arthralgias | no | no | no |
| | | knee AVN | | | | |
| Renal | Nephritis | hematuria | recurrent | no | n/a | nephrolithiasis |
| Involvement | | associated | nephrolithiasis | | | (no nephritis) |
| | | with episodes | (no nephritis) | | | |
| | | of alveolar | | | | |
| | | hemorrhage | | | | |
| | Proteinuria | no (mild | no | no (trace | n/a | no |
| | | elevations | | on UA) | | |
| | | <2x ULN) | | | | |
| | CKD | no | no | no | no | no |

| | | | | | |
|---|---|---|---|---|---|
| Chronic | yes | yes | no | no | no |
| immunosuppression | | | | | |
| Smoker | no | no | yes | yes | yes |

| Identifier | | B.II.1 | B.III.1 | B.I.2 | C.II.2 | C.I.1 |
|---|---|---|---|---|---|---|
| General | COPA | WT/R233H | WT/R233H | WT/R233H | WT/R233H | WT/R233H |
| | genotype | | | | | |
| | STING | 232H/232H | 232R/232H | HAQ/232H | 232H/232H | HAQ/232H |
| | genotype | | | | | |
| | Race, | white, non- | white, non- | white, non- | white, | white, |
| | ethnicity | Hispanic | Hispanic | Hispanic | Hispanic | Hispanic |
| | Status | alive | alive | alive | alive | alive |
| | Cause of | | | | | |
| | Death | | | | | |
| | Sex | male | female | female | female | male |
| Age | Symptom | 14 years | 6 months | n/a | 18 years | n/a |
| | onset | | | | | |
| | Last | 52 years | 7 years | 78 years | 24 years | 51 years |
| | evaluation | | | | | |
| | Death | | | | | |
| Preventing | Pulmonary | | + | | + | |
| symptoms | Joint | + | | | | |
| | FTT | | + | | | |
| Autoantibodies | ANA | 1:80, | 1:640, | 1:320, speckled | 1:1280 | neg |
| | | homogeneous & | homogeneous & | | homogeneous | |
| | | speckled | speckled, resolved | | | |
| | | | w treatment | | | |
| | ANCA/MPO/PR3 | pos; neg; neg | n/a; neg; neg | n/a; n/a; n/a | 1:320 (mostly | neg; n/a; n/a |
| | | | | | pANCA); | |
| | | | | | 207 U; neg | |
| | RF/CCP | 25 IU/mL | 1050 IU/mL; | neg; n/a | 344 IU; 27U | neg; neg |
| | | (IgA); neg | >250 U | | | |
| Pulmonary | Symptoms | yes | yes | none | yes | none |
| Involvement | Hemorrhage | no | yes | no | yes | no |
| | CT consistent | yes | suggestive of yes | no | yes | no |
| | With COPA | supportive | (CT at 16 mo) | no reticulation, | supportive | no reticulation, |
| | Syndrome | findings: | supportive findings: | traction | findings: GGOs, | traction |
| | | cystic changes, | GGOs, nodules | bronchiectasis, or | hemosiderin | bronchiectasis, or |
| | | honeycombing, | consistent with | cystic changes | deposition | cystic changes |
| | | traction | lymphocytic | | pattern, | |
| | | bronchiectasis, | interstitial | | cystic changes | |
| | | reticulation | pneumonia pattern | | | |
| | Transplant | no | no | not indicated | no | not indicated |
| | Biopsy | n/a | lymphocytic | not indicated | n/a | not indicated |
| | | | follicular | | | |
| | | | bronchiolitis, | | | |
| | | | minimal | | | |
| | | | capillaritis | | | |
| | | | (19 mo) | | | |
| MSK | Arthritis | yes | no | no | no | no |
| Involvement | Other | no | no | no | no | no |
| Renal | Nephritis | no | no | no | crescentic | no |
| Involvement | | | | | glomerulonephritis, | |
| | | | | | global and | |
| | | | | | segmental | |
| | | | | | glomerulosclerosis | |
| | Proteinuria | no | UPCR >1 | no | UPCR 1.1-1.6 | no |
| | | | intermittently | | from 4.6 initial | |
| | CKD | no | no | no | no | no |

| | | | | | |
|---|---|---|---|---|---|
| Chronic | | yes | no | yes | no |
| immunosuppression | | | | | |
| Smoker | no | no | no | no | no |

| Identifier | | E.II.1 | E.I.2 | G.II.4 | H.II.1 | H.I.2 |
|---|---|---|---|---|---|---|
| General | COPA | WT/V242D | WT/V239D | WT/D239G | WT/A239P | WT/A239P |
| | genotype | | | | | |
| | STING | 232H/232H | HAQ/232H | 232R/HAQ | 232R/232R | 232R/HAQ |
| | genotype | | | | | |
| | Race, | white, non- | white, non- | white, non- | east Asian | east Asian |
| | ethnicity | Hispanic | Hispanic | Hispanic | | |
| | Status | deceased | alive | alive | alive | alive |
| | Cause of | lung disease | | | | |
| | Death | | | | | |
| | Sex | female | female | male | male | female |
| Age | Symptom | 6 months | n/a | n/a | 4.5 years | |
| | onset | | | | | |
| | Last | 38 years | 74 years | 60 years | 8 years | 41 years |
| | evaluation | | | | | |
| | Death | 38 years | | | | |
| Presenting | Pulmonary | + | | | | |
| symptoms | Joint | + | | | | |
| | FTT | + | | | | |
| Autoantibodies | ANA | 1:1280 (unknown | 1:320, | n/a | 1:640, | no |
| | | pattern) | homogeneous | | homogeneous | |
| | | | | | and speckled | |
| | ANCA/MPO/PR3 | n/a; <0.2; <0.2 | 1:160 pANCA; | n/a; n/a; n/a | 1:20 atypical | no |
| | | | n/a; n/a | | pANCA; 60.4 | |
| | | | | | CU; 24.2 CU | |
| | RF/CCP | <10; 34 U | 113U; <0.5 | n/a; n/a | neg; neg | n/a; n/a |
| Pulmonary | Symptoms | yes | none | none | yes | none |
| Involvement | Hemorrhage | no | no | no | no | no |
| | CT | yes | no | n/a | yes | no |
| | consistent | supportive | no fibrosis | | supportive | |
| | with COPA | findings: GGOs, | on plain chest | | findings: | |
| | Syndrome | reticulation | radiograph | | cysts in a | |
| | | | | | subpleural | |
| | | | | | pattern | |
| | Transplant | combined heart/long | not indicated | not indicated | no | not indicated |
| | Biopsy | follicular | not indicated | not indicated | follicular | not indicated |
| | | bronchiolitis, | | | bronchiolitis | |
| | | pleural fibrosis, | | | | |
| | | hemosiderin laden | | | | |
| | | macrophages w/o | | | | |
| | | capillaritis (18 y) | | | | |
| MSK | Arthritis | yes | no | no | no | no |
| Involvement | Other | AVN | no | no | no | no |
| Renal | Nephritis | post-transplant; | n/a | n/a | no | n/a |
| Involvement | | aHUS in setting | | | | |
| | | of aspergillus | | | | |
| | Proteinuria | moderate on UA | n/a | n/a | no (mild | n/a |
| | | | | | elevations | |
| | | | | | <2x ULN) | |
| | CKD | required dialysis | no | no | no | no |

| | | | | | |
|---|---|---|---|---|---|
| Chronic | yes | no | no | yes | no |
| immunosuppression | | | | | |
| Smoker | n/a | n/a | no | no | no |

Footnotes: Chest imaging refers to computed tomography (CT) scans unless otherwise specified. Autoantibody titer of 1:80 or higher considered positive for ANA. Other musculoskeletal involvement refers to symptoms other than inflammatory arthritis. Proteinuria defined as 2 or more instances of urine protein creatinine ratio (UPCR) >1. Subject identifiers color coded in red for affected patients and blue for unaffected carriers. Pathogenic COPA allele noted in red, protective HAQ STING1 allele highlighted in blue. Abbreviations: neg=evaluated and negative; n/a=has not been evaluated; MSK=musculoskeletal; FTT=failure to thrive; ANA=antinuclear antibody; ANCA=antineutrophil cytoplasmic antibody; MPG=myeloperoxidase antibody; PR3=proteinase 3 antibody; RF=Rheumatoid Factor; CCP=anti-cyclic citrullinated peptide antibody; CKD=chronic kidney disease, defined by glomerular filtration rate below 60; 11D=interstitial lung disease; GGO=ground glass opacities; AVN=avascular necrosis; UPCR=urine protein to creatinine ratio; ULN=upper limit ofnormal; UA=urinalysis; aHUS=atypical hemolytic uremic syndrome

TABLE 3
Subject identification in prior publications

| Subject | Clinical Status | Prior Publication | Individual ID |
|---|---|---|---|
| A.III.3 | affected patient | (Watkin et al., 2015) | Family C III.3 |
| A.IV.1 | affected patient | (Watkin et al., 2015) | Family C IV.1 |
| A.IV.2 | affected patient | (Watkin et al., 2015) | Family C IV.2 |
| A.IV.8 | affected patient | (Watkin et al., 2015) | Family C IV.15 |
| A.V.1 | affected patient | (Watkin et al., 2015) | Family C V.1 |
| A.V.3 | affected patient | (Watkin et al., 2015) | Family C V.3 |
| B.II.1 | affected patient | None | |
| B.III.1 | affected patient | None | |
| C.II.1 | affected patient | (Cabrera-Pérez | |
| | | et al., 2022) | |
| C.II.2 | affected patient | (Cabrera-Pérez | |
| | | et al., 2022) | |
| D.II.1 | affected patient | (Volpi et al., 2018) | |
| E.II.1 | affected patient | None | |
| E.II.2 | affected patient | None | |
| F.III.5 | affected patient | (Watkin et al., 2015) | Family D II.4 |
| F.II.5 | affected patient | (Watkin et al., 2015) | Family D III.5 |
| G.II.2 | affected patient | (Watkin et al., 2015) | Family B II.2 |
| G.II.3 | affected patient | (Watkin et al., 2015) | Family B II.3 |
| G.II.6 | affected patient | (Watkin et al., 2015) | Family B II.6 |
| G.III.1 | affected patient | (Watkin et al., 2015) | Family B III.1 |
| G.III.3 | affected patient | (Watkin et al., 2015) | Family B III.3 |
| H.II.1 | affected patient | (Psarianos et al., 2021) | |
| I.I.2 | affected patient | (Watkin et al., 2015) | Family E I.2 |
| I.II.1 | affected patient | (Watkin et al., 2015) | Family E II.1 |
| sporadic 1 | affected patient | (Thaivalappil | |
| | | et al., 2021) | |
| sporadic 2 | affected patient | None | |
| sporadic 3 | affected patient | (Fagundes et al., 2024) | |
| A.III.2 | unaffected carrier | (Watkin et al., 2015) | Family C III.2 |
| A.III.5 | unaffected carrier | (Watkin et al., 2015) | Family C III.9 |
| A.IV.6 | unaffected carrier | (Watkin et al., 2015) | Family C IV.6 |
| B.I.2 | unaffected carrier | None | |
| C.I.1 | unaffected carrier | (Cabrera-Pérez | |
| | | et al., 2022) | |
| D.I.2 | unaffected carrier | (Volpi et al., 2018) | |
| E.I.2 | unaffected carrier | None | |
| G.II.4 | unaffected carrier | (Watkin et al., 2015) | Family B II.4 |
| H.I.2 | unaffected carrier | (Psarianos et al., 2021) | |

TABLE 4
Interferon score per study subject

| | | PBMC Interferon | On Immunosuppressive |
|---|---|---|---|
| | | Score (oldest to | Therapy at Sample |
| Subject | Clinical Status | newest) | Collection |
| A.IV.1 | affected patient | 275.5, 28.6 | yes, yes |
| A.IV.2 | affected patient | 248, 41.6 | yes |
| A.IV.8 | affected patient | 53.1, 101.6 | yes |
| A.V.1 | affected patient | 98.6 | yes |
| C.II.2 | affected patient | 9.6, 16.7 | yes, yes |
| C.II.3 | affected patient | 12.2, 9.2 | yes, yes |
| D.II.1 | affected patient | 16.7, 12.3 | interruption, yes |
| E.II.1 | affected patient | 21.5 | yes |
| G.III.3 | affected patient | 8.1 | yes |
| H.II.1 | affected patient | 88.4 | yes |
| I.I.2 | affected patient | 25 | yes |
| I.II.1 | affected patient | 67.5 | yes |
| A.III.2 | unaffected carrier | 1.6, 1.6 | no |
| A.IV.6 | unaffected carrier | 2.6 | no |
| B.I.2 | unaffected carrier | 16.1 | no |
| C.I.1 | unaffected carrier | 1.3 | no |
| D.I.2 | unaffected carrier | 0.7 | no |
| E.I.2 | unaffected carrier | 13.9 | no |
| G.II.4 | unaffected carrier | 0.64 | no |
| H.I.2 | unaffected carrier | 15.9 | no |
| | healthy control | 0.3 | no |
| | healthy control | 0.4 | no |
| | healthy control | 0.5 | no |
| | healthy control | 0.6 | no |
| | healthy control | 0.7 | no |
| | healthy control | 0.9 | no |
| | healthy control | 1 | no |
| | healthy control | 1 | no |
| | healthy control | 1 | no |
| | healthy control | 1.1 | no |
| | healthy control | 1.2 | no |
| | healthy control | 1.3 | no |
| | healthy control | 1.7 | no |
| | healthy control | 2.4 | no |
| | healthy control | 4.5 | no |

TABLE 5
Serum interferon activity per subject

| | | | On |
|---|---|---|---|
| | | | Immunosuppressive |
| | | Serum | Therapy |
| | | Interferon | at Sample |
| Subject | Clinical Status | Activity | Collection |
| A.IV.1 | affected patient | 3.4, 4.3 | yes, yes |
| A.IV.2 | affected patient | 7, 15.9 | yes, yes |
| A.IV.8 | affected patient | 1.2, 1.8, 4.8 | yes, yes, yes |
| A.V.1 | affected patient | 9.2, 5.4, 6.2, 0.2 | yes, yes, yes, yes |
| B.II.1 | affected patient | 1.6 | yes |
| B.III.1 | affected patient | 3 | yes |
| G.III.1 | affected patient | 5 | yes |
| H.II.1 | affected patient | 5.3 | yes |
| sporadic 2 | affected patient | 4.1 | yes |
| A.III.2 | unaffected carrier | 1.1, 3.6 | no, no |
| A.IV.6 | unaffected carrier | 2.5, 3.3 | no, no |
| B.I.2 | unaffected carrier | 1.8 | no |
| G.II.4 | unaffected carrier | 2.2 | no |
| H.I.2 | unaffected carrier | 0.6 | no |
| | healthy control | 0.2 | no |
| | healthy control | 0.3 | no |
| | healthy control | 0.9 | no |
| | healthy control | 1 | no |
| | healthy control | 1.4 | no |
| | healthy control | 1.8 | no |
| | healthy control | 1.9 | no |
| | healthy control | 2.1 | no |
| | healthy control | 2.2 | no |
| | healthy control | 3.2 | no |
| | healthy control | 3.7 | no |
| | healthy control | 4 | no |

TABLE 6
Primers used herein

| Purpose | Primer | Sequence |
|---|---|---|
| E241K CRISPR | Guide | UUCAGAAUCAAAGGCAUGGG |
| construct | RNA | |
| | HDR | TCCTCAGAATTGCTGAGGATCAACTCTTGGCGAGG |
| | template | GTGGAAGACGGCACAAGATACATTGTTGTAATGGC |
| | | CCCGGCAGGTATCAACTTTCCAGGCTTTTGATTCTG |
| | | AAGGACAAAAAGAATTAGGTC |
| Murine Cc15 | Forward | ACCACTCCCTGCTGCTTTGCCT |
| quantitative PCR | Reverse | GGCACACACTTGGCGGTTCCTT |
| Murine Cxc110 | Forward | AGTGCTGCCGTCATTTTCTGCCTC |
| quantitative PCR | Reverse | GCAGGATAGGCTCGCAGGGATGATT |
| Murine GAPDH | Forward | AGGTCGGTGTGAACGGATTTG |
| quantitative PCR | Reverse | TGTAGACCATGTAGTTGAGGTCA |

### REFERENCES

Adli, A. D. F., R. Jahanban-Esfahlan, K. Seidi, S. Samandari-Rad, and N. Zarghami. 2018. An overview on Vadimezan (DMXAA): The vascular disrupting agent. Chem. Biol. Drug Des. 91:996-1006. doi:10.1111/cbdd.13166.
Amouzegar, A., M. Chelvanambi, J. N. Filderman, W. J. Storkus, and J. J. Luke. 2021. STING Agonists as Cancer Therapeutics. Cancers. 13:2695. doi:10.3390/cancers13112695.
Arboleda-Velasquez, J. F., F. Lopera, M. O'Hare, S. Delgado-Tirado, C. Marino, N. Chmielewska, K. L. Saez-Torres, D. Amarnani, A. P. Schultz, R. A. Sperling, D. Leyton-Cifuentes, K. Chen, A. Baena, D. Aguillon, S. Rios-Romenets, M. Giraldo, E. Guzmán-Vélez, D. J. Norton, E. Pardilla-Delgado, A. Artola, J. S. Sanchez, J. Acosta-Uribe, M. Lalli, K. S. Kosik, M. J. Huentelman, H. Zetterberg, K. Blennow, R. A. Reiman, J. Luo, Y. Chen, P. Thiyyagura, Y. Su, G. R. Jun, M. Naymik, X. Gai, M. Bootwalla, J. Ji, L. Shen, J. B. Miller, L. A. Kim, P. N. Tariot, K. A. Johnson, E. M. Reiman, and Y. T. Quiroz. 2019. Resistance to autosomal dominant Alzheimer's disease in an APOE3 Christchurch homozygote: a case report. Nat. Med. 25:1680-1683. doi:10.1038/s41591-019-0611-3.
Cabrera-Pérez, J. S., J. Branch, A. Reyes, M. Michael, K. W. Eldin, M. Silva-Carmona, and T. P. Vogel. 2022. A Zebra at the Rodeo: Dyspnea, Hematuria, and a Family History of Arthritis. Arthrit Care Res. 74:165-170. doi:10.1002/acr.24368.
Cerboni, S., N. Jeremiah, M. Gentili, U. Gehrmann, C. Conrad, M.-C. Stolzenberg, C. Picard, B. Neven, A. Fischer, S. Amigorena, F. Rieux-Laucat, and N. Manel. 2017. Intrinsic antiproliferative activity of the innate sensor STING in T lymphocytes. J Exp Med. 214:1769-1785. doi:10.1084/jem.20161674.
Claussnitzer, M., J. H. Cho, R. Collins, N.J. Cox, E. T. Dermitzakis, M. E. Hurles, S. Kathiresan, E. E. Kenny, C. M. Lindgren, D. G. MacArthur, K. N. North, S. E. Plon, H. L. Rehm, N. Risch, C. N. Rotimi, J. Shendure, N. Soranzo, and M. I. McCarthy. 2020. A brief history of human disease genetics. Nature. 577:179-189. doi:10.1038/s41586-019-1879-7.
Cooper, D. N., M. Krawczak, C. Polychronakos, C. Tyler-Smith, and H. Kehrer-Sawatzki. 2013. Where genotype is not predictive of phenotype: towards an understanding of the molecular basis of reduced penetrance in human inherited disease. Hum. Genet. 132:1077-1130. doi:10.1007/s00439-013-1331-2.
Decout, A., J. D. Katz, S. Venkatraman, and A. Ablasser. 2021. The cGAS-STING pathway as a therapeutic target in inflammatory diseases. Nat Rev Immunol. 21:548-569. doi:10.1038/s41577-021-00524-z.
Deng, Z., Z. Chong, C. S. Law, K. Mukai, F. O. Ho, T. Martinu, B. J. Backes, W. L. Eckalbar, T. Taguchi, and A. K. Shum. 2020. A defect in COPI-mediated transport of STING causes immune dysregulation in COPA syndrome. J Exp Med. 217: e20201045. doi:10.1084/jem.20201045.
Fagundes, M. da C., T. Bianco, D. P. Nunes, T. K. D. Ostroski, G. das P. Bridi, A. M. Kawassaki, C. S. V. Barbas, L. O. Mendonga, S. F. Barros, J. Kalil, A. K. Shum, and D. L. Escuissato. 2024. Twenty-year-old patient with polyarthritis since childhood showing cysts and ground glass attenuation on HRCT. Thorax. thorax-2023-220798. doi:10.1136/thorax-2023-220798.
Feng, S., S. Sekine, V. Pessino, H. Li, M. D. Leonetti, and B. Huang. 2017. Improved split fluorescent proteins for endogenous protein labeling. Nat Commun. 8:370. doi:10.1038/s41467-017-00494-8.
Frémond, M.-L., and N. Nathan. 2021. COPA syndrome, 5 years after: Where are we?Joint Bone Spine. 88:105070. doi:10.1016/j.jbspin.2020.09.002.
Froechlich, G., A. Finizio, A. Napolano, S. Amiranda, A. D. Chiara, P. Pagano, M. Mallardo, G. Leoni, N. Zambrano, and E. Sasso. 2023. The common H232 STING allele shows impaired activities in DNA sensing, susceptibility to viral infection, and in monocyte cell function, while the HAQ variant possesses wild-type properties. Sci. Rep. 13:19541. doi:10.1038/s41598-023-46830-5.
Gruber, C., and D. Bogunovic. 2020. Incomplete penetrance in primary immunodeficiency: a skeleton in the closet. Hum. Genet. 139:745-757. doi:10.1007/s00439-020-02131-9.
Haellman, V., T. Strittmatter, A. Bertschi, P. Stücheli, and M. Fussenegger. 2021. A versatile plasmid architecture for mammalian synthetic biology (VAMSyB). Metab Eng. 66:41-50. doi:10.1016/j.ymben.2021.04.003.
Jeltema, D., K. Abbott, and N. Yan. 2023. STING trafficking as a new dimension of immune signaling. J Exp Med. 220: e20220990. doi:10.1084/jem.20220990.
Jin, L., L. Xu, I. V. Yang, E. J. Davidson, D. A. Schwartz, M. M. Wurfel, and J. C. Cambier. 2011. Identification and characterization of a loss-of-function human MPYS variant. Genes Immun. 12:263-269. doi:10.1038/gene.2010.75.
Kato, T., M. Yamamoto, Y. Honda, T. Orimo, I. Sasaki, K. Murakami, H. Hemmi, Y. Fukuda-Ohta, K. Isono, S. Takayama, H. Nakamura, Y. Otsuki, T. Miyamoto, J. Takita, T. Yasumi, R. Nishikomori, T. Matsubayashi, K. Izawa, and T. Kaisho. 2021. Augmentation of Stimulator of Interferon Genes-Induced Type I Interferon Production in COPA Syndrome. Arthritis Rheumatol. 73:2105-2115. doi:10.1002/art.41790.
Kemmoku, H., K. Takahashi, K. Mukai, T. Mori, K. M. Hirosawa, F. Kiku, Y. Uchida, Y. Kuchitsu, Y. Nishioka, M. Sawa, T. Kishimoto, K. Tanaka, Y. Yokota, H. Arai, K. G. N. Suzuki, and T. Taguchi. 2024. Single-molecule localization microscopy reveals STING clustering at the trans-Golgi network through palmitoylation-dependent accumulation of cholesterol. Nat. Commun. 15:220. doi:10.1038/s41467-023-44317-5.
Kingdom, R., and C. F. Wright. 2022. Incomplete Penetrance and Variable Expressivity: From Clinical Studies to Population Cohorts. Front. Genet. 13:920390. doi:10.3389/fgene.2022.920390.
Kuchitsu, Y., K. Mukai, R. Uematsu, Y. Takaada, A. Shinojima, R. Shindo, T. Shoji, S. Hamano, E. Ogawa, R. Sato, K. Miyake, A. Kato, Y. Kawaguchi, M. Nishitani-Isa, K. Izawa, R. Nishikomori, T. Yasumi, T. Suzuki, N. Dohmae, T. Uemura, G. N. Barber, H. Arai, S. Waguri, and T. Taguchi. 2023. STING signalling is terminated through ESCRT-dependent microautophagy of vesicles originating from recycling endosomes. Nat. Cell Biol. 25:453-466. doi:10.1038/s41556-023-01098-9.
Lepelley, A., M. J. Martin-Niclós, M. L. Bihan, J. A. Marsh, C. Uggenti, G. I. Rice, V. Bondet, D. Duffy, J. Hertzog, J. Rehwinkel, S. Amselem, S. B.-E. Khalifi, M. Brennan, E. Carter, L. Chatenoud, S. Chhun, A. C. l'Hermine, M. Depp, M. Legendre, K. J. Mackenzie, J. Marey, C. McDougall, K. J. McKenzie, T. J. Molina, B. Neven, L. Seabra, C. Thumerelle, M. Wislez, N. Nathan, N. Manel, Y. J. Crow, and M.-L. Fremond. 2020. Mutations in COPA lead to abnormal trafficking of STING to the Golgi and interferon signaling. J Exp Med. 217: e20200600. doi:10.1084/jem.20200600.
Meier, H. C. S., D. P. Sandler, E. M. Simonsick, N. Weng, and C. G. Parks. 2020. Sex differences in the association between antinuclear antibody positivity with diabetes and multimorbidity in older adults: Results from the Baltimore Longitudinal Study of Aging. Exp. Gerontol. 135:110906. doi:10.1016/j. exger.2020.110906.
Meric-Bernstam, F., R. F. Sweis, F. S. Hodi, W. A. Messersmith, R. H. I. Andtbacka, M. Ingham, N. Lewis, X. Chen, M. Pelletier, X. Chen, J. Wu, T. W. Dubensky, S. M. McWhirter, T. Müller, N. Nair, and J. J. Luke. 2021. Phase I Dose-Escalation Trial of MIW815 (ADU-S100), an Intratumoral STING Agonist, in Patients With Advanced/Metastatic Solid Tumors or Lymphomas. Clin. Cancer Res. 28:clincanres. CCR-21-1963-E.2021. doi:10.1158/1078-0432.ccr-21-1963.
Meric-Bernstam, F., R. F. Sweis, S. Kasper, O. Hamid, S. Bhatia, R. Dummer, A. Stradella, G. V. Long, A. Spreafico, T. Shimizu, N. Steeghs, J. J. Luke, S. M. McWhirter, T. Müller, N. Nair, N. Lewis, X. Chen, A. Bean, L. Kattenhorn, M. Pelletier, and S. Sandhu. 2022. Combination of the STING Agonist MIW815 (ADU-S100) and PD-1 Inhibitor Spartalizumab in Advanced/Metastatic Solid Tumors or Lymphomas: An Open-Label, Multicenter, Phase Ib Study. Clin. Cancer Res. 29:110-121. doi:10.1158/1078-0432.ccr-22-2235.
Mukai, K., E. Ogawa, R. Uematsu, Y. Kuchitsu, F. Kiku, T. Uemura, S. Waguri, T. Suzuki, N. Dohmae, H. Arai, A. K. Shum, and T. Taguchi. 2021. Homeostatic regulation of STING by retrograde membrane traffic to the ER. Nat Commun. 12:61. doi:10.1038/s41467-020-20234-9.
Pan, B.-S., S. A. Perera, J. A. Piesvaux, J. P. Presland, G. K. Schroeder, J. N. Cumming, B. W. Trotter, M. D. Altman, A. V. Buevich, B. Cash, S. Cemerski, W. Chang, Y. Chen, P. J. Dandliker, G. Feng, A. Haidle, T. Henderson, J. Jewell, I. Kariv, I. Knemeyer, J. Kopinja, B. M. Lacey, J. Laskey, C. A. Lesburg, R. Liang, B. J. Long, M. Lu, Y. Ma, E. C. Minnihan, G. O'Donnell, R. Otte, L. Price, L. Rakhilina, B. Sauvagnat, S. Sharma, S. Tyagarajan, H. Woo, D. F. Wyss, S. Xu, D. J. Bennett, and G. H. Addona. 2020. An orally available non-nucleotide STING agonist with antitumor activity. Science. 369. doi:10.1126/science.aba6098.
Patel, S., S. M. Blaauboer, H. R. Tucker, S. Mansouri, J. S. Ruiz-Moreno, L. Hamann, R. R. Schumann, B. Opitz, and L. Jin. 2017. The Common R71H-G230A-R293Q Human TMEM173 Is a Null Allele. J Immunol. 198:776-787. doi:10.4049/jimmunol.1601585.
Patel, S., and L. Jin. 2019. TMEM173 variants and potential importance to human biology and disease. Genes Immun. 20:82-89. doi:10.1038/s41435-018-0029-9.
Psarianos, P., J. Y. Y. Kwan, S. Dell, W. B. Wee, K. Rey-McIntyre, H. Chen, D. Dissanayake, R. M. Laxer, A. Shum, F.-F. Liu, and K. W. Yip. 2021. COPA Syndrome (Ala239Pro) Presenting with Isolated Follicular Bronchiolitis in Early Childhood: Case Report. J Clin Immunol. 41:1660-1663. doi:10.1007/s10875-021-01082-8.
Rahit, K. M. T. H., and M. Tarailo-Graovac. 2020. Genetic Modifiers and Rare Mendelian Disease. Genes. 11:239. doi:10.3390/genes11030239.
Ramanjulu, J. M., G. S. Pesiridis, J. Yang, N. Concha, R. Singhaus, S.-Y. Zhang, J.-L. Tran, P. Moore, S. Lehmann, H. C. Eberl, M. Muelbaier, J. L. Schneck, J. Clemens, M. Adam, J. Mehlmann, J. Romano, A. Morales, J. Kang, L. Leister, T. L. Graybill, A. K. Charnley, G. Ye, N. Nevins, K. Behnia, A. I. Wolf, V. Kasparcova, K. Nurse, L. Wang, A. C. Puhl, Y. Li, M. Klein, C. B. Hopson, J. Guss, M. Bantscheff, G. Bergamini, M. A. Reilly, Y. Lian, K. J. Duffy, J. Adams, K. P. Foley, P. J. Gough, R. W. Marquis, J. Smothers, A. Hoos, and J. Bertin. 2018. Design of amidobenzimidazole STING receptor agonists with systemic activity. Nature. 564:439-443. doi:10.1038/s41586-018-0705-y.
Reus, J. B., G. S. Trivino-Soto, L. I. Wu, K. Kokott, and E. S. Lim. 2020. SV40 Large T Antigen Is Not Responsible for the Loss of STING in 293T Cells but Can Inhibit cGAS-STING Interferon Induction. Viruses. 12:137. doi:10.3390/v12020137.
Rice, G. I., G. M. A. Forte, M. Szynkiewicz, D. S. Chase, A. Aeby, M. S. Abdel-Hamid, S. Ackroyd, R. Allcock, K. M. Bailey, U. Balottin, C. Barnerias, G. Bernard, C. Bodemer, M. P. Botella, C. Cereda, K. E. Chandler, L. Dabydeen, R. C. Dale, C. D. Laet, C. G. E. L. D. Goede, M. del Toro, L. Effat, N. N. Enamorado, E. Fazzi, B. Gener, M. Haldre, J.-P. S.-M. Lin, J. H. Livingston, C. M. Lourenco, W. Marques, P. Oades, P. Peterson, M. Rasmussen, A. Roubertie, J. L. Schmidt, S. A. Shalev, R. Simon, R. Spiegel, K. J. Swoboda, S. A. Temtamy, G. Vassallo, C. N. Vilain, J. Vogt, V. Wermenbol, W. P. Whitehouse, D. Soler, I. Olivieri, S. Orcesi, M. S. Aglan, M. S. Zaki, G. M. H. Abdel-Salam, A. Vanderver, K. Kisand, F. Rozenberg, P. Lebon, and Y. J. Crow. 2013. Assessment of interferon-related biomarkers in Aicardi-Goutieres syndrome associated with mutations in TREX1, RNASEH2A, RNASEH2B, RNASEH2C, SAMHD1, and ADAR: a case-control study. Lancet Neurol. 12:1159-1169. doi:10.1016/s1474-4422(13)70258-8.
Rohm, F., E. Kling, R. Hoffmann, C. Meisinger, and J. Linseisen. 2024. Prevalence of a large panel of systemic autoantibodies in the Bavarian adult population. Front. Immunol. 15:1355905. doi:10.3389/fimmu.2024.1355905.
Ruiz-Moreno, J. S., L. Hamann, J. A. Shah, A. Verbon, F. P. Mockenhaupt, M. Puzianowska-Kuznicka, J. Naujoks, L. E. Sander, M. Witzenrath, J. C. Cambier, N. Suttorp, R. R. Schumann, L. Jin, T. R. Hawn, B. Opitz, and C. S. Group. 2018. The common HAQ STING variant impairs cGAS-dependent antibacterial responses and is associated with susceptibility to Legionnaires' disease in humans. Plos Pathog. 14: e1006829. doi:10.1371/journal.ppat.1006829.
Shang, G., C. Zhang, Z. J. Chen, X. Bai, and X. Zhang. 2019. Cryo-EM structures of STING reveal its mechanism of activation by cyclic GMP-AMP. Nature. 567:389-393. doi:10.1038/s41586-019-0998-5.
Simchoni, N., T. P. Vogel, and A. K. Shum. 2023. COPA Syndrome from Diagnosis to Treatment A Clinician's Guide. Rheum. Dis. Clin. North Am. 49:789-804. doi:10.1016/j.rdc.2023.06.005.
Sivick, K. E., N. H. Surh, A. L. Desbien, E. P. Grewal, G. E. Katibah, S. M. McWhirter, and T. W. Dubensky. 2017. Comment on “The Common R71H-G230A-R293Q Human TMEM173 Is a Null Allele.” J Immunol. 198:4183-4185. doi:10.4049/jimmunol.1700294.
   Steiner, A., K. Hrovat-Schaale, I. Prigione, C.-H. Yu, P. Laohamonthonkul, C. R. Harapas, R. R. J. Low, D. D. Nardo, L. F. Dagley, M. J. Mlodzianoski, K. L. Rogers, T. Zillinger, G. Hartmann, M. P. Gantier, M. Gattorno, M. Geyer, S. Volpi, S. Davidson, and S. L. Masters. 2022. Deficiency in coatomer complex I causes aberrant activation of STING signalling. Nat Commun. 13:2321. doi:10.1038/s41467-022-29946-6.
Thaivalappil, S. S., A. S. Garrod, S. M. Borowitz, L. B. Watkin, and M. G. Lawrence. 2021. Persistent Unexplained Transaminitis in COPA Syndrome. J Clin Immunol. 41:205-208. doi:10.1007/s10875-020-00832-4.
Thiele, H., and P. Nurnberg. 2005. HaploPainter: a tool for drawing pedigrees with complex haplotypes. Bioinformatics. 21:1730-1732. doi:10.1093/bioinformatics/bth488.
Tsui, J. L., O. A. Estrada, Z. Deng, K. M. Wang, C. S. Law, B. M. Elicker, K. D. Jones, S. D. Dell, G. Gudmundsson, S. Hansdottir, S. M. Helfgott, S. Volpi, M. Gattorno, M. R. Waterfield, A. Y. Chan, S. A. Chung, B. Ley, and A. K. Shum. 2018. Analysis of pulmonary features and treatment approaches in the COPA syndrome. Erj Open Res. 4:00017-02018. doi:10.1183/23120541.00017-2018.
Volpi, S., J. Tsui, M. Mariani, C. Pastorino, R. Caorsi, O. Sacco, A. Ravelli, A. K. Shum, M. Gattorno, and P. Picco. 2018. Type I interferon pathway activation in COPA syndrome. Clin Immunol. 187:33-36. doi:10.1016/j.clim.2017.10.001.
Watkin, L. B., B. Jessen, W. Wiszniewski, T. J. Vece, M. Jan, Y. Sha, M. Thamsen, R. L. P. Santos-Cortez, K. Lee, T. Gambin, L. R. Forbes, C. S. Law, A. Stray-Pedersen, M. H. Cheng, E. M. Mace, M. S. Anderson, D. Liu, L. F. Tang, S. K. Nicholas, K. Nahmod, G. Makedonas, D. L. Canter, P.-Y. Kwok, J. Hicks, K. D. Jones, S. Penney, S. N. Jhangiani, M. D. Rosenblum, S. D. Dell, M. R. Waterfield, F. R. Papa, D. M. Muzny, N. Zaitlen, S. M. Leal, C. Gonzaga-Jauregui, E. Boerwinkle, N. T. Eissa, R. A. Gibbs, J. R. Lupski, J. S. Orange, and A. K. Shum. 2015. COPA mutations impair ER-Golgi transport and cause hereditary autoimmune-mediated lung disease and arthritis. Nat Genet. 47:654-660. doi:10.1038/ng.3279.
Yang, Y., D. M. Muzny, F. Xia, Z. Niu, R. Person, Y. Ding, P. Ward, A. Braxton, M. Wang, C. Buhay, N. Veeraraghavan, A. Hawes, T. Chiang, M. Leduc, J. Beuten, J. Zhang, W. He, J. Scull, A. Willis, M. Landsverk, W. J. Craigen, M. R. Bekheirnia, A. Stray-Pedersen, P. Liu, S. Wen, W. Alcaraz, H. Cui, M. Walkiewicz, J. Reid, M. Bainbridge, A. Patel, E. Boerwinkle, A. L. Beaudet, J. R. Lupski, S. E. Plon, R. A. Gibbs, and C. M. Eng. 2014. Molecular Findings Among Patients Referred for Clinical Whole-Exome Sequencing. JAMA. 312:1870-1879. doi:10.1001/jama.2014.14601.
Yi, G., V. P. Brendel, C. Shu, P. Li, S. Palanathan, and C. C. Kao. 2013. Single Nucleotide Polymorphisms of Human STING Can Affect Innate Immune Response to Cyclic Dinucleotides. Plos One. 8: e77846. doi:10.1371/journal.pone.0077846.

### Example 2: Similarities Between SAVI and COPA Syndrome

STING-associated vasculopathy with onset in infancy (SAVI) is an interferonopathy similar to COPA syndrome.

| COPA syndrome | SAVI |
|---|---|
| Type I IFN signature | Type I IFN signature |
| Autosomal dominant inheritance | Autosomal dominant inheritance |
| Interstitial lung disease | Interstitial lung disease |
| Capillaritis (pulmonary) | Capillaritis (skin) |
| Defect in transport | Constitutive exit of |
| between Golgi and ER | STING from ER to Golgi |

INFORMAL SEQUENCE LISTING

| SEQ ID NO: | Name of sequence | Sequence |
|---|---|---|
| 1. | E241K CRISPR | UUCAGAAUCAAAGGCAUGGG |
| | construct guide | |
| | RNA | |
| 2. | E241K CRISPR | TCCTCAGAATTGCTGAGGATCAACTCTTGGCGAGGG |
| | construct HDR | TGGAAGACGGCACAAGATACATTGTTGTAATGGCC |
| | template | CCGGCAGGTATCAACTTTCCAGGCTTTTGATTCTGA |
| | | AGGACAAAAAGAATTAGGTC |
| 3. | Murine Ccl5 | ACCACTCCCTGCTGCTTTGCCT |
| | quantitative PCR | |
| | Forward | |
| 4. | Murine Cel5 | GGCACACACTTGGCGGTTCCTT |
| | quantitative PCR | |
| | Reverse | |
| 5. | Murine Cxcl10 | AGTGCTGCCGTCATTTTCTGCCTC |
| | quantitative PCR | |
| | Forward | |
| 6. | Murine Cxcl10 | GCAGGATAGGCTCGCAGGGATGATT |
| | quantitative PCR | |
| | Reverse | |
| 7. | Murine GAPDH | AGGTCGGTGTGAACGGATTTG |
| | quantitative PCR | |
| | Forward | |
| 8. | Murine GAPDH | TGTAGACCATGTAGTTGAGGTCA |
| | quantitative PCR | |
| | Reverse | |

What is claimed is:

1. A method of treating a Stimulator of Interferon Genes (STING)-mediated disease in a subject having or being at risk of having coatomer protein subunit alpha (COPA) syndrome, said method comprising administering a therapeutically effective amount of a STING gene therapy to said subject thereby treating said STING-mediated disease in said subject.

2. The method of claim 1, wherein said STING gene therapy comprises gene transfer, gene addition, gene replacement, or genome editing.

3. The method of claim 2, wherein said STING gene therapy comprises introducing a histidine at a position corresponding to position 71 of STING, an alanine at a position corresponding to position 230 of STING, and a glutamine at a position corresponding to position 293 of STING to said subject.

4. The method of claim 2, wherein said STING gene therapy comprises expressing a recombinant nucleic acid encoding a histidine at a position corresponding to position 71 of STING, an alanine at a position corresponding to position 230 of STING, and a glutamine at a position corresponding to position 293 of STING in said subject.

5. The method of claim 3, wherein said recombinant nucleic acid forms part of a lentiviral vector.

6. The method of claim 5, wherein said STING gene therapy comprises expressing a minor allele of STING.

7. The method of claim 6, wherein said minor allele comprises rs11554776, rs78233829 and rs7380824.

8. The method of claim 7, wherein said STING gene therapy comprises expressing a recombinant nucleic acid sequence encoding rs11554776, rs78233829 and rs7380824 single nucleotide polymorphisms (SNPs) in said subject.

9. The method of claim 8, wherein said recombinant nucleic acid sequence forms part of a lentiviral vector.

10. The method of claim 9, wherein said STING-mediated diseases is a monogenic autoinflammatory syndrome, an autoimmune disease, a neurological disorder, a metabolic disease, an inflammatory disease, a cardiovascular disease or cancer.

11. The method of claim 10, wherein said STING-mediated diseases is Aicardi Goutieres syndrome (AGS), or Familial chilblain lupus.

12. The method of claim 10, wherein said STING-mediated diseases is systemic lupus erythematosus or rheumatoid arthritis.

13. The method of claim 10, wherein said STING-mediated diseases is an ischaemic brain injury, Parkinson disease, general neurodegeneration, Huntington disease, amyotrophic lateral sclerosis, frontotemporal dementia, age-dependent macular degeneration or traumatic brain injury.

14. The method of claim 10, wherein said STING-mediated diseases is nonalcoholic steatohepatitis, alcoholic liver disease or acute pancreatitis.

15. The method of claim 10, wherein said STING-mediated diseases is silica-induced fibrosis or sepsis.

16. The method of claim 10, wherein said STING-mediated diseases is myocardial infarction or chronic heart failure.

17. The method of claim 10, wherein said STING-mediated diseases is colorectal cancer, skin cancer or metastases.

18. The method of claim 10, wherein said subject is a senescent subject.

19. The method of claim 10, wherein said subject is not a STING-associated vasculopathy (SAVI) subject.

20. The method of claim 1, said method comprising prior to said administering a therapeutically effective amount of a STING gene therapy to said subject, detecting an arginine at a position corresponding to position 232 of STING, a histidine at a position corresponding to position 232, a glycine at a position corresponding to position 230 of STING, an arginine at a position corresponding to position 293 of STING, or an arginine at a position corresponding to position 71 of STING.

21. The method of claim 20, wherein the presence of said arginine at a position corresponding to position 232 of STING, said histidine at a position corresponding to position 232, said glycine at a position corresponding to position 230 of STING, said arginine at a position corresponding to position 293 of STING, or said arginine at a position corresponding to position 71 of STING indicates said subject has or is at risk of developing a STING-mediated disease.

22. The method of claim 1, said method comprising prior to said administering a therapeutically effective amount of a STING gene therapy to said subject, detecting a rs1131769 SNP in said subject.

23. The method of claim 22, wherein the presence of said rs1131769 SNP indicates said subject has or is at risk of developing a STING-mediated disease.
